(19) United States
(12) Patent Application Publication
(10) Pub. No.: US 2019/0228844 A1
(43) Pub. Date: Jul. 25, 2019

(54) EVALUATING METHOD, EVALUATING APPARATUS, EVALUATING PROGRAM PRODUCT, EVALUATING SYSTEM, AND TERMINAL APPARATUS FOR COLORECTAL CANCER

(71) Applicant: AJINOMOTO CO., INC., Chuo-ku (JP)

(72) Inventors: Hidehiro NAKAMURA, Kanagawa (JP); Natsumi NISHIKATA, Kanagawa (JP); Akira IMAIZUMI, Kanagawa (JP); Kazutaka SHIMBO, Kanagawa (JP); Naoko ARASHIDA, Kanagawa (JP); Rumi NISHIMOTO, Kanagawa (JP)

(73) Assignee: AJINOMOTO CO., INC., Chuo-ku (JP)

(21) Appl. No.: 16/373,427

(22) Filed: Apr. 2, 2019

Related U.S. Application Data
(63) Continuation of application No. PCT/JP2017/036199, filed on Oct. 4, 2017.

(30) Foreign Application Priority Data
Oct. 4, 2016 (JP) 2016-196712

(51) Int. Cl. G16C 20/70

(52) U.S. Cl. CPC G16C 20/70

(57) ABSTRACT

An evaluating method includes an evaluating step of evaluating a state of colorectal cancer for a subject to be evaluated using (i) a concentration value of at least one metabolite of bABA, N-Me-bABA, 1-Me-His, aABA, Aminoadipic acid, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, SDMA, Acylcarnitine (13:1), and EPA in blood of the subject or (ii) a value of a formula calculated using the concentration value of the metabolite and the formula including an explanatory variable to be substituted with the concentration value of the metabolite.

### CROSS-REFERENCE TO RELATED APPLICATION

This application is based upon and claims the benefit of priority from PCT Application PCT/JP2017/036199, filed Oct. 4, 2017, which claims priority from Japanese Patent Application No. 2016-196712, filed Oct. 4, 2016, the entire contents of which are incorporated herein by reference.

### BACKGROUND OF THE INVENTION

### 1. Field of the Invention

The present invention relates to an evaluating method, an evaluating apparatus, an evaluating program product, an evaluating system, and a terminal apparatus.

### 2. Description of the Related Art

The number of deaths from colorectal cancer in Japan in 2003 is 21962 males and 18373 females, and the number of deaths from colorectal cancer ranks fourth in males among the deaths from cancers, while the number of deaths from colorectal cancer ranks first in females among the deaths from cancers. The morbidity rate of colorectal cancer in recent years tends to be leveling off.

The five-year survival rate of early colorectal cancer (A and B in Dukes classification) is 80% or higher, and particularly, the five-year survival rate of class A colorectal cancer (tumor remaining within the colorectal wall) is about 90%.

However, the five-year survival rate of progressive colorectal cancer, particularly in class D of Dukes classification (having distal metastasis to the peritoneum, liver, lung and the like) is extremely lowered to about 10%. Therefore, early detection is important for the healing of colorectal cancer.

Here, diagnosis of colorectal cancer includes diagnosis based on the immunological fecal occult blood reaction, diagnosis by colonoscopy, and the like.

However, diagnosis based on a fecal occult blood test does not serve as definitive diagnosis, and most of the persons with positive-finding are false-positive. Furthermore, in regard to early colorectal cancer, there is a concern that both the detection sensitivity and the detection specificity become lower in the diagnosis based on a fecal occult blood test or the diagnosis by colonoscopy. In particular, early cancer in the right side colon is frequently overlooked when diagnosed by a fecal occult blood test. Diagnostic imaging by CT (computer tomography), MRI (magnetic resonance imaging), PET (positron emission computerized-tomography) or the like is not suitable for the diagnosis of colorectal cancer.

On the other hand, colorectal biopsy by colonoscopy serves as definitive diagnosis, but is a highly invasive examination, and implementing colonoscopic examination at the screening stage is not practical. Furthermore, invasive diagnosis such as colonoscopy gives a burden to patients, such as accompanying pain, and there may also be a risk of bleeding upon examination, or the like.

Therefore, from the viewpoints of a physical burden imposed on patients and of cost-benefit performance, it is desirable to narrow down the target range of test subjects with high possibility of onset of colorectal cancer, and to subject those people to treatment. Specifically, it is desirable that test subjects are selected by a less invasive method, the target range of the selected test subjects is narrowed by subjecting the selected test subjects to a colonoscopic examination, and the test subjects who are definitively diagnosed as having colorectal cancer are subjected to treatment.

Incidentally, it is known that the concentrations of amino acids in blood change as a result of onset of cancer. For example, Cynober (“Cynober, L. ed., Metabolic and therapeutic aspects of amino acids in clinical nutrition. 2nd ed., CRC Press.”) has reported that, for example, the amount of consumption increases in cancer cells, for glutamine mainly as an oxidation energy source, for arginine as a precursor of nitrogen oxide and polyamine, and for methionine through the activation of the ability of cancer cells to take in methionine, respectively. Vissers, et al. (“Vissers, Y. LJ., et. al., Plasma arginine concentration are reduced in cancer patients: evidence for arginine deficiency?, The American Journal of Clinical Nutrition, 2005. 81, p 1142-1146”) and Park (“Park, K. G., et. al., Arginine metabolism in benign and maglinant disease of breast and colon: evidence for possible inhibition of tumor-infiltrating macropharges, Nutrition, 1991. 7, p. 185-188”) have reported that the amino acid composition in plasma in colorectal cancer patients is different from that of healthy individuals.

WO 2004/052191, WO 2006/098192, and WO 2009/054351 related to a method of relating an amino acid concentration and a biological state are disclosed as previous patents. WO 2008/016111 related to a method of evaluating a state of lung cancer using an amino acid concentration, WO 2008/075662 related to a method of evaluating a state of breast cancer using an amino acid concentration, WO 2008/075663 related to a method of evaluating a state of colorectal cancer using an amino acid concentration, WO 2008/075664 related to a method of evaluating a state of cancer using an amino acid concentration, WO 2009/099005 related to a method of evaluating a state of gastric cancer using an amino acid concentration, WO 2009/110517 related to a method of evaluating a cancer type using an amino acid concentration, WO 2009/154296 related to a method of evaluating a state of female genital cancer using an amino acid concentration, WO 2009/154297 related to a method of evaluating a state of prostatic disease including at least one of prostatic cancer and prostatic hypertrophy using an amino acid concentration, WO 2014/084290 related to a method of evaluating a state of pancreatic cancer using an amino acid concentration, and JP-A-2014-106114 related to a method of evaluating a state of pancreatic cancer risk disease using an amino acid concentration are disclosed as previous patents.

In the meantime, with respect to metabolites having a blood concentration lower than that of amino acids, development of measurement instruments such as a LC-MS and a LC-MS/MS is revealing that the blood concentration of the metabolites in the blood of a lung-cancer patient varies. For example, according to WO 2011/096210, it has been reported that an ADMA concentration in blood serum of a lung-cancer patient increases. According to JP-A-2011-247869, it has been reported that a sarcosine concentration in blood serum of a lung-cancer patient increases.

However, there is a problem that the development of techniques of diagnosing colorectal cancer with metabolites in blood as tumor markers is not conducted or not practically used.

### SUMMARY OF THE INVENTION

It is an object of the present invention to at least partially solve the problems in the conventional technology.

The present invention has been made in view of the above descriptions, and an object of the present invention is to provide an evaluating method, an evaluating apparatus, an evaluating program product, an evaluating system, and a terminal apparatus, which can provide reliable information that may be helpful in knowing a state of colorectal cancer.

To solve the problem and achieve the object described above, an evaluating method according to one aspect of the present invention includes an evaluating step of evaluating a state of colorectal cancer for a subject to be evaluated using a concentration value of at least one of 25 kinds of metabolites (1-Me-His (1-methyl-histidine), aABA (α-aminobutyric acid), Aminoadipic acid (α-aminoadipic acid), bABA (β-aminobutyric acid), bAiBA (β-amino-iso-butyric acid), Cadaverine, Ethylglycine, GABA (γ-aminobutyric acid), Homoarginine, Hypotaurine, Kinurenine, N6-Acetyl-L-Lys (N6-Acetyl-L-Lysine), Putrescine, Serotonin, Spermidine, Spermine, ADMA (asymmetric dimethylarginine), Homocitrulline, 3-Me-His (3-methyl-histidine), Hydroxyproline, Phosphoethanolamine, SDMA (symmetric dimethylarginine), N-Me-bABA (N-methyl-β-aminobutyric acid), AC (13:1) (Acylcarnitine (13:1)), and EPA (cis-5,8,11,14,17-Eicosapentaenoic acid)) in blood of the subject.

The evaluating method according to another aspect of the present invention is the evaluating method, wherein the evaluating step further uses a concentration value of at least one of 19 kinds of amino acids (Asn, His, Thr, Ala, Cit, Arg, Tyr, Val, Met, Lys, Trp, Gly, Pro, Orn, Ile, Leu, Phe, Ser, and Gln) in blood of the subject.

In the present description, various amino acids are mainly written in abbreviations, the formal names of these are as follows.

| (Abbreviation) | (Formal name) |
|---|---|
| Ala | Alanine |
| Arg | Arginine |
| Asn | Asparagine |
| Cit | Citrulline |
| Gln | Glutamine |
| Gly | Glycine |
| His | Histidine |
| Ile | Isoleucine |
| Leu | Leucine |
| Lys | Lysine |
| Met | Methionine |
| Orn | Ornithine |
| Phe | Phenylalanine |
| Pro | Proline |
| Ser | Serine |
| Thr | Threonine |
| Trp | Tryptophan |
| Tyr | Tyrosine |
| Val | Valine |

The evaluating method according to still another aspect of the present invention is the evaluating method, wherein the evaluating step evaluates the state of colorectal cancer for the subject by calculating a value of a formula further using the formula including an explanatory variable to be substituted with the concentration value of at least one of the 25 kinds of metabolites.

The evaluating method according to still another aspect of the present invention is the evaluating method, wherein the evaluating step further uses the concentration value of at least one of the 19 kinds of amino acids in blood of the subject, and the formula further includes an explanatory variable to be substituted with the concentration value of at least one of the 19 kinds of amino acids.

An evaluating apparatus according to one aspect of the present invention is an evaluating apparatus including a control unit. The control unit includes an evaluating unit that evaluates a state of colorectal cancer for a subject to be evaluated using a concentration value of at least one of the 25 kinds of metabolites in blood of the subject.

An evaluating method according to one aspect of the present invention is an evaluating method executed by an information processing apparatus including a control unit. The evaluating method includes an evaluating step of evaluating a state of colorectal cancer for a subject to be evaluated using a concentration value of at least one of the 25 kinds of metabolites in blood of the subject. The evaluating step is executed by the control unit.

An evaluating program product according to one aspect of the present invention is an evaluating program product having a non-transitory tangible computer readable medium including programmed instructions for causing an information processing apparatus including a control unit to execute an evaluating method. The evaluating method includes an evaluating step of evaluating a state of colorectal cancer for a subject to be evaluated using a concentration value of the 25 kinds of metabolites in blood of the subject. The evaluating step is executed by the control unit.

A recording medium according to one aspect of the present invention is a non-transitory tangible computer-readable recording medium including programmed instructions for causing an information processing apparatus to execute the evaluating method.

An evaluating system according to one aspect of the present invention is an evaluating system including an evaluating apparatus including a control unit and a terminal apparatus including a control unit to provide concentration data on a concentration value of at least one of the 25 kinds of metabolites in blood of a subject to be evaluated. The evaluating apparatus and the terminal apparatus are connected to each other communicatively via a network. The control unit of the terminal apparatus includes a concentration data-sending unit that transmits the concentration data of the subject to the evaluating apparatus and a result-receiving unit that receives an evaluation result on a state of colorectal cancer for the subject transmitted from the evaluating apparatus. The control unit of the evaluating apparatus includes a concentration data-receiving unit that receives the concentration data of the subject transmitted from the terminal apparatus, an evaluating unit that evaluates the state of colorectal cancer for the subject using the concentration value of at least one of the 25 kinds of metabolites included in the concentration data of the subject received by the concentration data-receiving unit, and a result-sending unit that transmits the evaluation result obtained by the evaluating unit to the terminal apparatus.

A terminal apparatus according to one aspect of the present invention is a terminal apparatus including a control unit. The control unit includes a result-obtaining unit that obtains an evaluation result on a state of colorectal cancer for a subject to be evaluated. The evaluation result is the result of evaluating the state of colorectal cancer for the subject using a concentration value of at least one of the 25 kinds of metabolites in blood of the subject.

The terminal apparatus according to another aspect of the present invention is the terminal apparatus, wherein the apparatus is communicatively connected via a network to an evaluating apparatus that evaluates the state of colorectal cancer for the subject. The control unit further includes a concentration data-sending unit that transmits concentration data on the concentration value of at least one of the 25 kinds of metabolites in blood of the subject to the evaluating apparatus. The result-obtaining unit receives the evaluation result transmitted from the evaluating apparatus.

An evaluating apparatus according to one aspect of the present invention is an evaluating apparatus including a control unit, being connected communicatively via a network to a terminal apparatus that provides concentration data on a concentration value of at least one of the 25 kinds of metabolites in blood of a subject to be evaluated. The control unit includes a concentration data-receiving unit that receives the concentration data of the subject transmitted from the terminal apparatus, an evaluating unit that evaluates a state of colorectal cancer for the subject using the concentration value of at least one of the 25 kinds of metabolites included in the concentration data of the subject received by the concentration data-receiving unit, and a result-sending unit that transmits an evaluation result obtained by the evaluating unit to the terminal apparatus.

According to the present invention, the state of colorectal cancer for the subject is evaluated using the concentration value of at least one of the 25 kinds of metabolites in blood of the subject. Thus, the present invention achieves the effect of being able to provide reliable information that may be helpful in knowing the state of colorectal cancer.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of a first embodiment;

FIG. 2 is a principle configurational diagram showing a basic principle of a second embodiment;

FIG. 3 is a diagram showing an example of an entire configuration of a present system;

FIG. 4 is a diagram showing another example of an entire configuration of the present system;

FIG. 5 is a block diagram showing an example of a configuration of an evaluating apparatus 100 in the present system;

FIG. 6 is a chart showing an example of information stored in a concentration data file 106a;

FIG. 7 is a chart showing an example of information stored in an index state information file 106b;

FIG. 8 is a chart showing an example of information stored in a designated index state information file 106c;

FIG. 9 is a chart showing an example of information stored in a formula file 106d1;

FIG. 10 is a chart showing an example of information stored in an evaluation result file 106e;

FIG. 11 is a block diagram showing a configuration of an evaluating part 102d;

FIG. 12 is a block diagram showing an example of a configuration of a client apparatus 200 in the present system; and

FIG. 13 is a block diagram showing an example of a configuration of a database apparatus 400 in the present system.

### DETAILED DESCRIPTION OF THE PREFERRED EMBODIMENTS

Hereinafter, an embodiment (first embodiment) of the evaluating method according to the present invention and an embodiment (second embodiment) of the evaluating apparatus, the evaluating method, the evaluating program product, the recording medium, the evaluating system, and the terminal apparatus according to the present invention are described in detail with reference to the drawings. The present invention is not limited to these embodiments.

### First Embodiment

1-1. Outline of First Embodiment

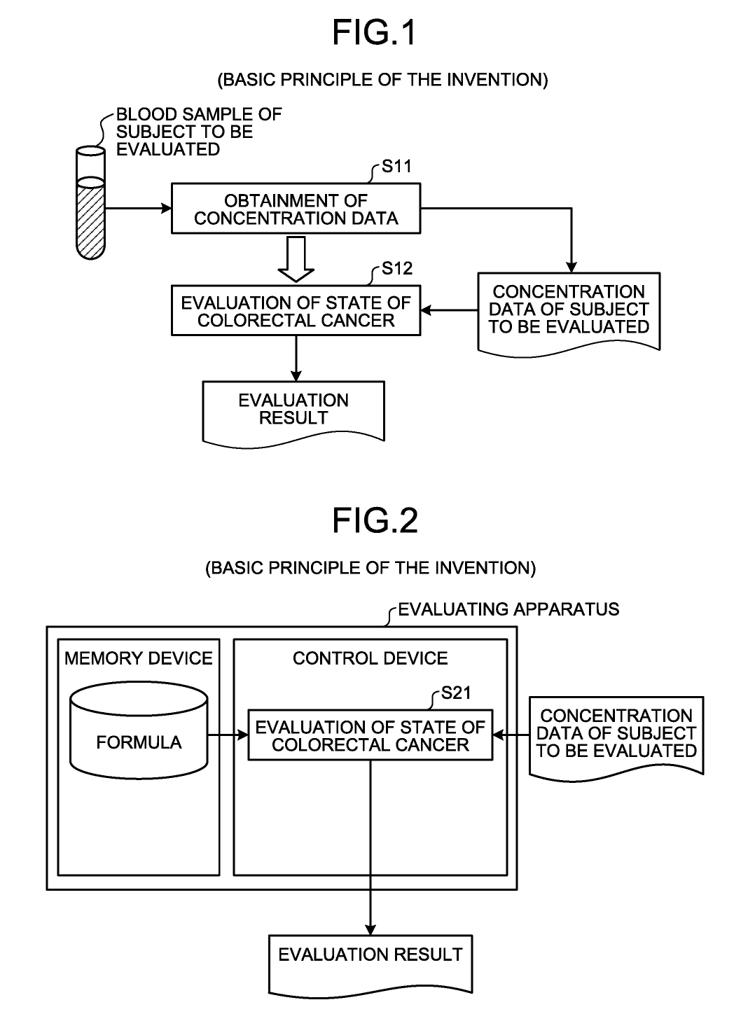

Here, an outline of the first embodiment will be described with reference to FIG. 1. FIG. 1 is a principle configurational diagram showing a basic principle of the first embodiment.

First, concentration data on a concentration value of a substance (a substance in blood including at least one of “the 25 kinds of metabolites and the 19 kinds of amino acids”) contained in blood (including, for example, plasma or serum) extracted from a subject to be evaluated (for example, an individual such as animal or human) is obtained (Step S11).

At step S11, for example, the concentration data on the substance in blood measured by a company or other organization that measures concentrations may be obtained. In addition, for example, the following measuring method of (A), (B), or (C) may be used to measure the concentration of the substance in blood from the blood sampled from the subject to obtain the concentration data on the concentration of the substance in blood. Here, the unit of the concentrations of the substances in blood may be molar concentration, weight concentration, enzyme activity, or one obtained by addition, subtraction, multiplication, and division of any constant with these concentrations.

(A) Plasma is separated from blood by centrifuging the collected blood sample. All plasma samples are frozen and stored at −80° C. until the concentration is measured. At the time of measuring the concentration, acetonitrile is added to deproteinize the plasma samples, pre-column derivatization is then performed using a labeling reagent (3-aminopyridyl-N-hydroxysuccinimidyl carbamate), and the concentration is analyzed by liquid chromatograph mass spectrometer (LC/MS) (see International Publication WO 2003/069328 and International Publication WO 2005/116629). Alternatively, phospholipid is removed from the deproteinized plasma by solid phase extraction, and then the concentarion (peak area) is analyzed by LC/MS.

(B) Plasma is separated from blood by centrifuging the collected blood sample. All plasma samples are frozen and stored at −80° C. until the concentration is measured. At the time of measuring the concentration, sulfosalicylic acid is added to deproteinize the plasma samples, and the concentration is analyzed by an amino acid analyzer based on post-column derivatization using a ninhydrin reagent.

(C) Blood cell separation is performed on the collected blood sample by using a membrane, MEMS (Micro Electro Mechanical Systems) technology, or the principle of centrifugation, whereby plasma or serum is separated from the blood. A plasma or serum sample the concentration of which is not measured immediately after obtaining the plasma or the serum is frozen and stored at −80° C. until the concentration is measured. At the time of measuring the concentration, a molecule that reacts with or binds to a target substance in blood, such as an enzyme or an aptamer, and the like are used to perform quantitative analysis and the like on an increasing or decreasing substance or a spectroscopic value by substrate recognition, whereby the concentration is analyzed.

The state of colorectal cancer for the subject is evaluated using the concentration value of at least one of the 25 kinds of metabolites and the 19 kinds of amino acids included in the concentration data obtained at step S11 (step S12). Before step S12 is executed, data such as defective and outliers may be removed from the concentration data obtained at step S11. Evaluation of a state is, for example, to examine the present condition.

According to the first embodiment described above, the concentration data of the subject is obtained at step S11, and at step S12, the state of colorectal cancer for the subject is evaluated using the concentration value of at least one of the 25 kinds of metabolites and the 19 kinds of amino acids included in the concentration data of the subject obtained at step S11. Hence, reliable information that may be helpful in knowing the state of colorectal cancer can be provided.

It may be decided that the concentration value of at least one of the 25 kinds of metabolites and the 19 kinds of amino acids reflects the state of colorectal cancer for the subject. The concentration value may be converted, for example, by the methods listed below, and it may be decided that the converted value reflects the state of colorectal cancer for the subject. In other words, the concentration value or the converted value may be treated per se as an evaluation result on the state of colorectal cancer for the subject.

The concentration value may be converted such that a possible range of the concentration value falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the concentration value, by conversion of the concentration value by a predetermined conversion method (for example, exponential transformation, logarithm transformation, angular transformation, square root transformation, probit transformation, reciprocal transformation, Box-Cox transformation, or power transformation), or by performing a combination of these computations on the concentration value. For example, a value of an exponential function with the concentration value as an exponent and Napier constant as the base may be further calculated (specifically, a value of p/(1−p) where a natural logarithm ln(p/(1−p)) is equal to the concentration value when the probability p that the state of colorectal cancer has a predetermined state (for example, a state of exceeding a criterion value and having colorectal cancer with a high probability) is defined), and a value (specifically, a value of the probability p) may be further calculated by dividing the calculated value of the exponential function by the sum of 1 and the value of the exponential function.

The concentration value may be converted such that the converted value is a particular value when a particular condition is met. For example, the concentration value may be converted such that the converted value is 5.0 when the specificity is 80% and the converted value is 8.0 when the specificity is 95%.

For each metabolite and each amino acid, after normally distributing the concentration distribution, the concentration value may be standardized with a mean of 50 and a standard deviation of 10.

These conversions may be performed by gender or age.

Positional information about a position of a predetermined mark on a predetermined scale visually presented on a display device such as a monitor or a physical medium such as paper may be generated using the concentration value of at least one of the 25 kinds of metabolites and the 19 kinds of amino acids or, if the concentration value is converted, the converted value, and it may be decided that the generated positional information reflects the state of colorectal cancer for the subject. The predetermined scale is for evaluating the state of colorectal cancer and is, for example, a graduated scale at least marked with graduations corresponding to the upper limit value and the lower limit value in “a possible range of the concentration value or the converted value, or part of the range”. The predetermined mark corresponds to the concentration value or the converted value and is, for example, a circle sign or a star sign.

If the concentration value of at least one of the 25 kinds of metabolites and the 19 kinds of amino acids is lower than a predetermined value (e.g., mean±1SD, 2SD, 3SD, N quantile, N percentile, or a cutoff value the clinical significance of which is recognized) or is equal to or lower than the predetermined value, or the concentration value is equal to or higher than the predetermined value or is higher than the predetermined value, the state of colorectal cancer for the subject may be evaluated. In this case, instead of the concentration value itself, a concentration standard score (a value obtained by normally distributing the concentration distribution by gender and then standardizing the concentration value with a mean of 50 and a standard deviation of 10 for each metabolite and each amino acid) may be used. For example, if the concentration standard score is lower than the mean −2SD (when the concentration standard score<30) or if the concentration standard score is higher than the mean+2SD (when the concentration standard score>70), the state of colorectal cancer for the subject may be evaluated.

The state of colorectal cancer for the subject may be evaluated by calculating a value of a formula using the concentration value of at least one of the 25 kinds of metabolites and the 19 kinds of amino acids and the formula including an explanatory variable to be substituted with the concentration value of at least one of the 25 kinds of metabolites and the 19 kinds of amino acids.

It may be decided that the calculated value of the formula reflects the state of colorectal cancer for the subject. The value of the formula may be converted, for example, by the methods listed below, and it may be decided that the converted value reflects the state of colorectal cancer for the subject. In other words, the value of the formula or the converted value may be treated per se as the evaluation result on the state of colorectal cancer for the subject.

The value of the formula may be converted such that a possible range of the value of the formula falls within the predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the value of the formula, by conversion of the value of the formula by the predetermined conversion method (for example, the exponential transformation, the logarithm transformation, the angular transformation, the square root transformation, the probit transformation, the reciprocal transformation, the Box-Cox transformation, or the power transformation), or by performing a combination of these computations on the value of the formula. For example, a value of an exponential function with the value of the formula as an exponent and Napier constant as the base may be further calculated (specifically, a value of p/(1−p) where a natural logarithm ln(p/(1−p)) is equal to the value of the formula when the probability p that the state of colorectal cancer has the predetermined state (for example, the state of exceeding the criterion value and having colorectal cancer with a high probability) is defined), and the value (specifically, the value of the probability p) may be further calculated by dividing the calculated value of the exponential function by the sum of 1 and the value of the exponential function.

The value of the formula may be converted such that the converted value is a particular value when a particular condition is met. For example, the value of the formula may be converted such that the converted value is 5.0 when the specificity is 80% and the converted value is 8.0 when the specificity is 95%.

The value of the formula may be standardized with a mean of 50 and a standard deviation of 10.

These conversions may be performed by gender or age.

The value of the formula in the present description may be the value of the formula per se or may be the converted value of the value of the formula.

The positional information about the position of the predetermined mark on the predetermined scale visually presented on the display device such as the monitor or the physical medium such as the paper may be generated using the value of the formula or, if the value of the formula is converted, the converted value, and it may be decided that the generated positional information reflects the state of colorectal cancer for the subject. The predetermined scale is for evaluating the state of colorectal cancer and is, for example, the graduated scale at least marked with the graduations corresponding to the upper limit value and the lower limit value in “a possible range of the value of the formula or the converted value, or part of the range”. The predetermined mark corresponds to the value of the formula or the converted value and is, for example, the circle sign or the star sign.

The degree of the possibility that the subject has colorectal cancer may be qualitatively evaluated. Specifically, the subject may be classified into any one of a plurality of categories defined at least considering the degree of the possibility of having colorectal cancer, using “the concentration value of at least one of the 25 kinds of metabolites and the 19 kinds of amino acids and one or more preset thresholds” or “the concentration value of at least one of the 25 kinds of metabolites and the 19 kinds of amino acids, the formula including the explanatory variable to be substituted with the concentration value of at least one of the 25 kinds of metabolites and the 19 kinds of amino acids, and one or more preset thresholds”. The categories may include a category to which a subject with a high degree of the possibility of having colorectal cancer (for example, a subject assumed to have colorectal cancer) belongs (for example, Rank C described in Examples), a category to which a subject with a low degree of the possibility of having colorectal cancer (for example, a subject assumed not to have colorectal cancer) belongs (for example, Rank A described in Examples), and a category to which a subject with an intermediate degree of the possibility of having colorectal cancer is belongs (for example, Rank B described in Examples). The categories may include the category to which the subject with a high degree of the possibility of having colorectal cancer belongs (for example, a colorectal cancer category described in Examples) and the category to which the subject with a low degree of the possibility of having colorectal cancer belongs (for example, a healthy category described in Examples to which the subject with a high possibility of being healthy (for example, the subject assumed to be healthy) belongs). The concentration value or the value of the formula may be converted by the predetermined method, and the subject may be classified into any one of the categories using the converted value.

As for the formula used for the evaluation, the form of the formula is not specifically designated, however, for example, may be the following forms.
linear model such as multiple regression equation, linear discriminant, principal component analysis, and canonical discriminant analysis that are based on the least-squares method;
generalized linear model such as logistic regression and Cox regression that are based on the maximum likelihood method;
generalized linear mixed model considering random effects due to individual differences, facility differences, and other factors in addition to the generalized linear model
expression generated by cluster analysis such as the K-means method and hierarchical cluster analysis;
expression generated on the basis of the Bayesian statistics such as the Markov chain Monte Carlo (MCMC), the Bayesian network, and the hierarchical Bayesian method;
expression generated by class classification such as support vector machine and decision tree;
expression generated by a method that does not belong to the above-cited categories such as fractional expression; and
expression represented as, for example, the summation of expressions of different forms

The formula used for the evaluation may be prepared by a method described in WO 2004/052191 that is an international application filed by the present applicant or by a method described in WO 2006/098192 that is an international application filed by the present applicant. Any formulae obtained by these methods can be preferably used in the evaluation of the state of colorectal cancer, regardless of the units of the concentrations of any one or both of the metabolites and the amino acids in the concentration data as input data.

In the multiple regression equation, the multiple logistic regression equation, and the canonical discriminant function, a coefficient and a constant term are added to each explanatory variable, and the coefficient and the constant term may be preferably real numbers, more preferably values in the range of 99% confidence interval for the coefficient and the constant term obtained from data for the various kinds of classifications described above, more preferably values in the range of 95% confidence interval for the coefficient and the constant term obtained from data for the various kinds of classifications described above. The value of each coefficient and the confidence interval thereof may be those multiplied by a real number, and the value of the constant term and the confidence interval thereof may be those having an arbitrary actual constant added or subtracted or those multiplied or divided by an arbitrary actual constant. When an expression such as the logistic regression, the linear discriminant, and the multiple regression equation is used for the evaluation, a linear transformation of the expression (addition of a constant and multiplication by a constant) and a monotonic increasing (decreasing) transformation (for example, a logit transformation) of the expression do not alter evaluation performance and thus evaluation performance after transformation is equivalent to that before transformation. Therefore, the expression includes an expression that is subjected to the linear transformation and the monotonic increasing (decreasing) transformation.

In the fractional expression, the numerator of the fractional expression is expressed by the sum of the explanatory variables A, B, C etc. and the denominator of the fractional expression is expressed by the sum of the explanatory variables a, b, c etc. The fractional expression also includes the sum of the fractional expressions α, β, γ etc. (for example, α+β) having such constitution. The fractional expression also includes divided fractional expressions. The explanatory variables used in the numerator or denominator may have suitable coefficients respectively. The explanatory variables used in the numerator or denominator may appear repeatedly. Each fractional expression may have a suitable coefficient. A value of a coefficient for each explanatory variable and a value for a constant term may be any real numbers. In a fractional expression and the one in which explanatory variables in the numerator and explanatory variables in the denominator in the fractional expression are switched with each other, the positive and negative signs are generally reversed in correlation with objective explanatory variables, but because their correlation is maintained, the evaluation performance can be assumed to be equivalent. The fractional expression therefore also includes the one in which explanatory variables in the numerator and explanatory variables in the denominator in the fractional expression are switched with each other.

When the state of colorectal cancer is evaluated, a value related to other biological information (for example, values listed below) may further be used in addition to the concentration value of at least one of the 25 kinds of metabolites and the 19 kinds of amino acids. The formulae used for the evaluation may additionally include one or more explanatory variables to be substituted with the value related to the other biological information (for example, values listed below) in addition to the explanatory variable to be substituted with the concentration value of at least one of the 25 kinds of metabolites and the 19 kinds of amino acids.

1. Concentration values of metabolites in blood other than amino acids (e.g., amino acid metabolites, carbohydrates, and lipids), proteins, peptides, minerals, hormones, and the like.

2. Blood test values such as albumin, total protein, triglyceride (neutral fat), HbA1c, glycoalbumin, insulin resistance index, total cholesterol, LDL cholesterol, HDL cholesterol, amylase, total bilirubin, creatinine, estimated glomerular filtration rate (eGFR), uric acid, GOT (AST), GPT (ALT), GGTP (γ-GTP), glucose (glucose level), CRP (C-reactive protein), erythrocyte, hemoglobin, hematocrit, MCV, MC, MCHC, leucoc yte and the number of thrombocytes.

3. Values obtained from image information such as ultrasonic echo, X ray, CT (Computer Tomography), MRI (Magnetic Resonance Imaging), and endoscope image.

4. Values of biological indices such as age, height, weight, BMI, abdominal girth, systolic blood pressure, diastolic blood pressure, gender, smoking information, dietary information, drinking information, exercise information, stress information, sleeping information, family medical history information, and disease history information (for example, diabetes).

### Second Embodiment

2-1. Outline of the Second Embodiment

Here, outlines of the second embodiment will be described in detail with reference to FIG. 2. FIG. 2 is a principle configurational diagram showing a basic principle of the second embodiment. In the description of the present second embodiment, description duplicating that of the first embodiment is sometimes omitted. In particular, herein, when the state of colorectal cancer is evaluated, a case of using the value of the formula or the converted value thereof is described as one example. However, for example, the concentration value of at least one of “the 25 kinds of metabolites and the 19 kinds of amino acids” or the converted value thereof (for example, the concentration standard score) may be used.

A control device evaluates the state of colorectal cancer for the subject by calculating the value of the formula using (i) the concentration value of at least one of the 25 kinds of metabolites and the 19 kinds of amino acids included in the previously obtained concentration data of the subject (for example, an individual such as animal or human) on the concentration value of at least one of the 25 kinds of metabolites and the 19 kinds of amino acids in blood and (ii) the formula previously stored in a memory device including the explanatory variable to be substituted with the concentration value of at least one of the 25 kinds of metabolites and the 19 kinds of amino acids (step S21). Hence, reliable information that may be helpful in knowing the state of colorectal cancer can be provided.

The formula used at step S21 may be generated based on the formula-preparing processing (step 1 to step 4) described below. Here, the summary of the formula-preparing processing is described. The processing described below is merely one example, and the method of preparing the formula is not limited thereto.

First, the control device prepares a candidate formula (e.g., y=a₁x₁+a₂x₂+ . . . +aₙxₙ, y: index data, xᵢ: concentration data, aᵢ: constant, i=1, 2, . . . , n) based on a predetermined formula-preparing method from index state information (data such as defective and outliers may be removed from the index state information in advance) previously stored in the memory device containing the concentration data and index data on an index representing the state of colorectal cancer (step 1).

In step 1, a plurality of the candidate formulae may be prepared from the index state information by using a plurality of the different formula-preparing methods (including those for multivariate analysis such as the principal component analysis, the discriminant analysis, the support vector machine, the multiple regression analysis, the Cox regression analysis, the logistic regression analysis, the K-means method, the cluster analysis, and the decision tree). Specifically, a plurality of groups of the candidate formulae may be prepared simultaneously and concurrently by using a plurality of different algorithms with the index state information which is multivariate data composed of the concentration data and the index data obtained by analyzing blood obtained from a large number of healthy groups and colorectal cancer groups. For example, the two different candidate formulae may be formed by performing the discriminant analysis and the logistic regression analysis simultaneously with the different algorithms. Alternatively, the candidate formula may be formed by converting the index state information with the candidate formula prepared by performing the principal component analysis and then performing the discriminant analysis of the converted index state information. In this way, it is possible to finally prepare the most suitable formula.

The candidate formula prepared by the principal component analysis is a linear expression including each explanatory variable maximizing the variance of all concentration data. The candidate formula prepared by the discriminant analysis is a high-powered expression (including exponential and logarithmic expressions) including each explanatory variable minimizing the ratio of the sum of the variances in respective groups to the variance of all concentration data. The candidate formula prepared by using the support vector machine is a high-powered expression (including kernel function) including each explanatory variable maximizing the boundary between groups. The candidate formula prepared by using the multiple regression analysis is a high-powered expression including each explanatory variable minimizing the sum of the distances from all concentration data. The candidate formula prepared by using the Cox regression analysis is a linear model including a logarithmic hazard ratio, and is a linear expression including each explanatory variable with a coefficient thereof maximizing the likelihood of the linear model. The candidate formula prepared by using the logistic regression analysis is a linear model expressing logarithmic odds of probability, and a linear expression including each explanatory variable maximizing the likelihood of the probability. The K-means method is a method of searching k pieces of neighboring concentration data in various groups, designating the group containing the greatest number of the neighboring points as its data-belonging group, and selecting the explanatory variable that makes the group to which input concentration data belong agree well with the designated group. The cluster analysis is a method of clustering (grouping) the points closest in entire concentration data. The decision tree is a method of ordering explanatory variables and predicting the group of concentration data from the pattern possibly held by the higher-ordered explanatory variable.

Returning to the description of the formula-preparing processing, the control device verifies (mutually verifies) the candidate formula prepared in step 1 based on a particular verifying method (step 2). The verification of the candidate formula is performed on each other to each candidate formula prepared in step 1. In step 2, at least one of discrimination rate, sensitivity, specificity, information criterion, ROC_AUC (area under the curve in a receiver operating characteristic curve), and the like of the candidate formula may be verified by at least one of bootstrap method, holdout method, N-fold method, leave-one-out method, and the like. In this way, it is possible to prepare the candidate formula higher in predictability or reliability, by taking the index state information and the evaluation condition into consideration.

The discrimination rate is a rate in which a subject to be evaluated whose true state is negative (for example, the subject who does not have colorectal cancer) is correctly evaluated as being negative by the evaluation method according to the present embodiment and a subject to be evaluated whose true state is positive (for example, the subject who has colorectal cancer) is correctly evaluated as being positive by the evaluation method according to the present embodiment. The sensitivity is a rate in which a subject to be evaluated whose true state is positive is correctly evaluated as being positive by the evaluation method according to the present embodiment. The specificity is a rate in which a subject to be evaluated whose true state is negative is correctly evaluated as being negative by the evaluation method according to the present embodiment. The Akaike information criterion is a criterion representing how observation data agrees with a statistical model, for example, in the regression analysis, and it is determined that the model in which the value defined by “−2×(maximum log-likelihood of statistical model)+2×(the number of free parameters of statistical model)” is smallest is the best. ROC_AUC is defined as the area under the receiver operating characteristics curve (ROC) created by plotting (x, y)=(1-specificity, sensitivity) on two-dimensional coordinates. The value of ROC_AUC is 1 in perfect discrimination, and the closer this value is to 1, the higher the discriminative characteristic. The predictability is the average of discrimination rates, sensitivities, or specificities obtained by repeating the validation of the candidate formula. The robustness refers to the variance of discrimination rates, sensitivities, or specificities obtained by repeating the validation of the candidate formula.

Returning to the description of the formula-preparing processing, the control device selects a combination of the concentration data contained in the index state information used in preparing the candidate formula, by selecting an explanatory variable of the candidate formula based on a predetermined explanatory variable-selecting method (step 3). In step 3, the selection of the explanatory variable may be performed on each candidate formula prepared in step 1. In this way, it is possible to select the explanatory variable of the candidate formula properly. The step 1 is executed once again by using the index state information including the concentration data selected in step 3. In step 3, the explanatory variable of the candidate formula may be selected based on at least one of stepwise method, best path method, local search method, and genetic algorithm from the verification result obtained in step 2. The best path method is a method of selecting an explanatory variable by optimizing an evaluation index of the candidate formula while eliminating the explanatory variables contained in the candidate formula one by one.

Returning to the description of the formula-preparing processing, the control device prepares the formula used for the evaluation by repeatedly performing steps 1, 2 and 3, and based on the verification results thus accumulated, selecting the candidate formula used for the evaluation from the candidate formulae (step 4). In the selection of the candidate formula, there are cases where the optimum formula is selected from the candidate formulae prepared in the same formula-preparing method or the optimum formula is selected from all candidate formulae.

As described above, in the formula-preparing processing, the processing for the preparation of the candidate formulae, the verification of the candidate formulae, and the selection of the explanatory variables in the candidate formulae are performed based on the index state information in a series of operations in a systematized manner, whereby the formula most appropriate for evaluating the state of colorectal cancer can be prepared. In other words, in the formula-preparing processing, the concentration of the substance in blood including at least one of the 25 kinds of metabolites and the 19 kinds of amino aicds is used in multivariate statistical analysis, and for selecting the optimum and robust combination of the explanatory variables, the explanatory variable-selecting method is combined with cross-validation to extract the formula having high evaluation performance.

2-2. System Configuration

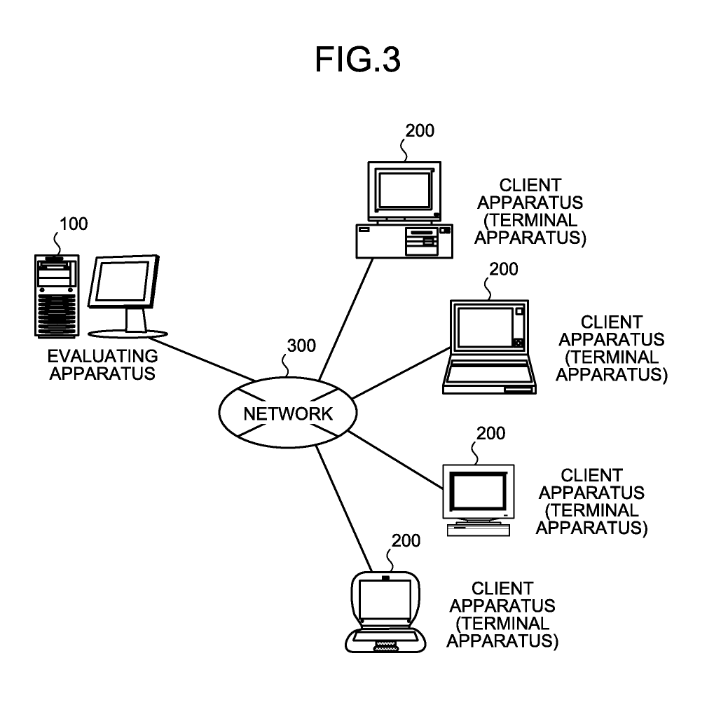

Hereinafter, the configuration of the evaluating system according to the second embodiment (hereinafter referred to sometimes as the present system) will be described with reference to FIGS. 3 to 14. This system is merely one example, and the present invention is not limited thereto. In particular, herein, when the state of colorectal cancer is evaluated, a case of using the value of the formula or the converted value thereof is described as one example. However, for example, the concentration value of at least one of “the 25 kinds of metabolites and the 19 kinds of amino acids” or the converted value thereof (for example, the concentration standard score) may be used.

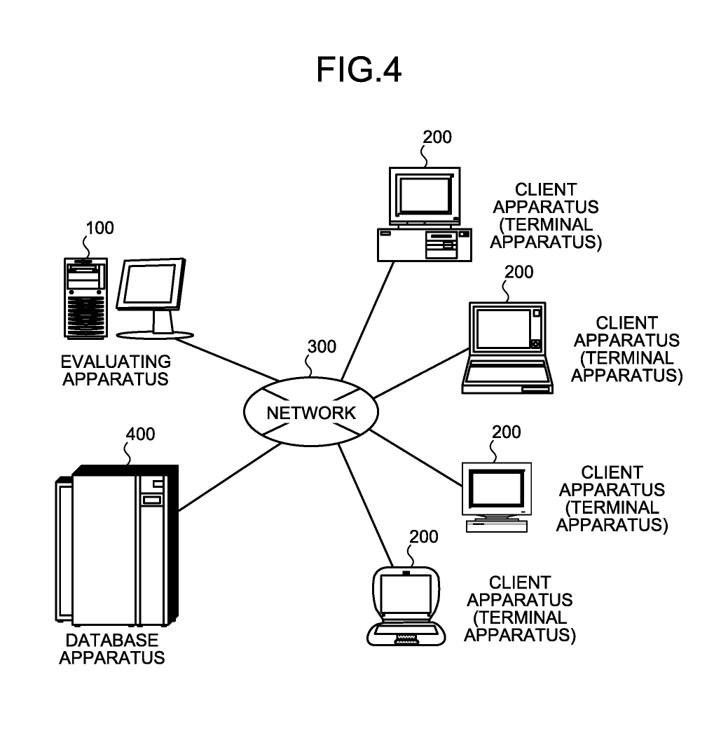

First, an entire configuration of the present system will be described with reference to FIGS. 3 and 4. FIG. 3 is a diagram showing an example of the entire configuration of the present system. FIG. 4 is a diagram showing another example of the entire configuration of the present system. As shown in FIG. 3, the present system is constituted in which the evaluating apparatus 100 that evaluates the state of colorectal cancer for the individual as the subject and the client apparatus 200 (corresponding to the terminal apparatus of the present invention) that provides the concentration data of the individual on the concentration value of the substance in blood including at least one of the 25 kinds of metabolites and the 19 kinds of amino acids in blood, are communicatively connected to each other via a network 300.

In the present system as shown in FIG. 4, in addition to the evaluating apparatus 100 and the client apparatus 200, the database apparatus 400 storing, for example, the index state information used in preparing the formula in the evaluating apparatus 100 and the formula used for the evaluation, may be communicatively connected via the network 300. In this configuration, for example, information that may be helpful in knowing the state of colorectal cancer is provided via the network 300 from the evaluating apparatus 100 to the client apparatuses 200 and the database apparatus 400, or from the client apparatuses 200 and the database apparatus 400 to the evaluating apparatus 100. The information that may be helpful in knowing the state of colorectal cancer is, for example, information on the measured value of a particular item as to the state of colorectal cancer of organisms including human. The information that may be helpful in knowing the state of colorectal cancer is generated in the evaluating apparatus 100, the client apparatus 200, or other apparatuses (e.g., various measuring apparatuses) and stored mainly in the database apparatus 400.

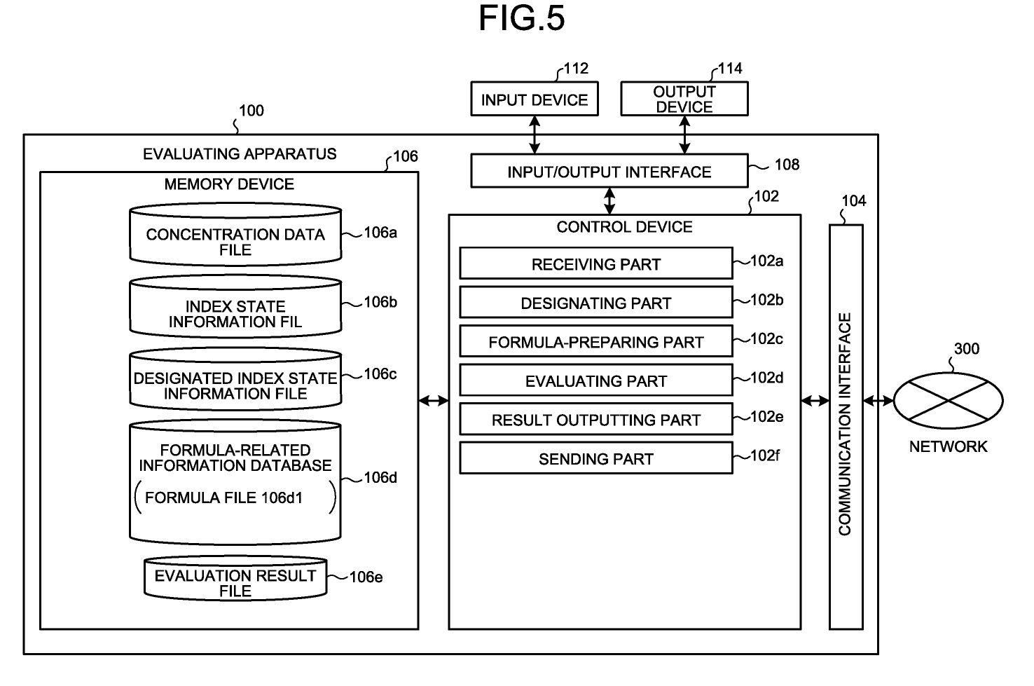

Now, the configuration of the evaluating apparatus 100 in the present system will be described with reference to FIGS. 5 to 11. FIG. 5 is a block diagram showing an example of the configuration of the evaluating apparatus 100 in the present system, showing conceptually only the region relevant to the present invention.

The evaluating apparatus 100 includes (I) a control device 102, such as CPU (Central Processing Unit), that integrally controls the evaluating apparatus, (II) a communication interface 104 that connects the evaluating apparatus to the network 300 communicatively via communication apparatuses such as a router and wired or wireless communication lines such as a private line, (III) a memory device 106 that stores various databases, tables, files and others, and (IV) an input/output interface 108 connected to an input device 112 and an output device 114, and these parts are connected to each other communicatively via any communication channel. The evaluating apparatus 100 may be present together with various analyzers (e.g., an amino acid analyzer) in a same housing. For example, the evaluating apparatus 100 may be a compact analyzing device including components (hardware and software) that calculate (measure) the concentration value of the predetermined substance in blood including at least one of the 25 kinds of metabolites and the 19 kinds of amino acids in blood and output (e.g., print or display on a monitor) the calculated value, wherein the compact analyzing device is characterized by further including the evaluating part 102d described later, and using the components to output results obtained by the evaluating part 102d.

The communication interface 104 allows communication between the evaluating apparatus 100 and the network 300 (or a communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as the monitor 114). A keyboard, a mouse, a microphone, or a monitor functioning as a pointing device together with a mouse may be used as the input device 112.

The memory device 106 is a storage means, and examples thereof include a memory apparatus such as RAM (Random Access Memory) and ROM (Read Only Memory), a fixed disk drive such as a hard disk, a flexible disk, and an optical disk. The memory device 106 stores computer programs giving instructions to the CPU for various processings, together with OS (Operating System). As shown in the figure, the memory device 106 stores the concentration data file 106a, the index state information file 106b, the designated index state information file 106c, a formula-related information database 106d, and the evaluation result file 106e.

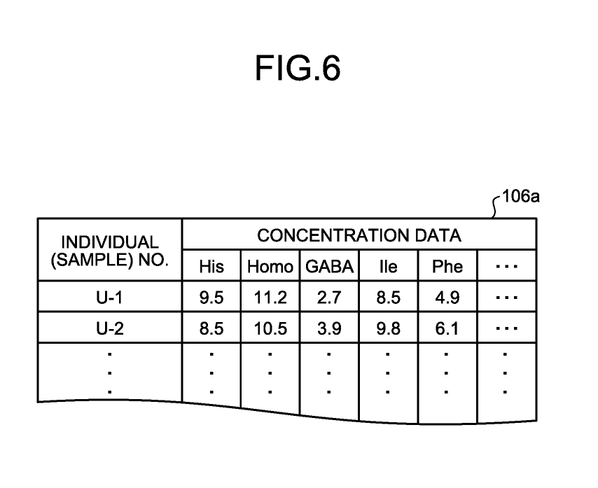

The concentration data file 106a stores the concentration data on the concentration value of the substance in blood including at least one of the 25 kinds of metabolites and the 19 kinds of amino acids in blood. FIG. 6 is a chart showing an example of information stored in the concentration data file 106a. As shown in FIG. 6, the information stored in the concentration data file 106a includes an individual number for uniquely identifying the individual (sample) as the subject and the concentration data that are correlated to one another. In FIG. 6, the concentration data is assumed to be numerical values, i.e., on a continuous scale, but the concentration data may be expressed on a nominal scale or an ordinal scale. In the case of the nominal or ordinal scale, any number may be allocated to each state for analysis. The concentration data may be combined with the value related to the other biological information (see above).

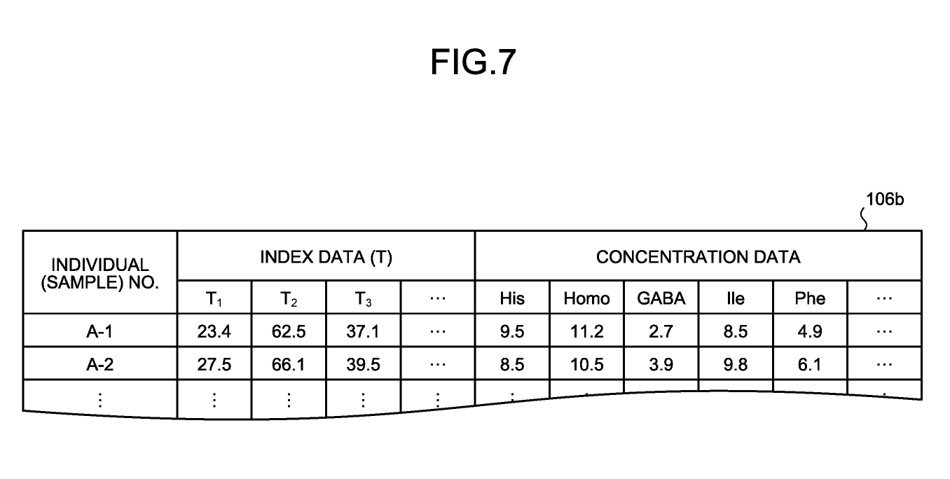

Returning to FIG. 5, the index state information file 106b stores the index state information used in preparing the formula. FIG. 7 is a chart showing an example of information stored in the index state information file 106b. As shown in FIG. 7, the information stored in the index state information file 106b includes the individual number, the index data (T) on the index representing the state of colorectal cancer (index T₁, index T₂, index T₃ . . . ), and the concentration data that are correlated to one another. In FIG. 7, the index data and the concentration data are assumed to be numerical values, i.e., on a continuous scale, but the index data and the concentration data may be expressed on a nominal scale or an ordinal scale. In the case of the nominal or ordinal scale, any number may be allocated to each state for analysis. The index data is, for example, a known index serving as a marker of the state of colorectal cancer, and numerical data may be used.

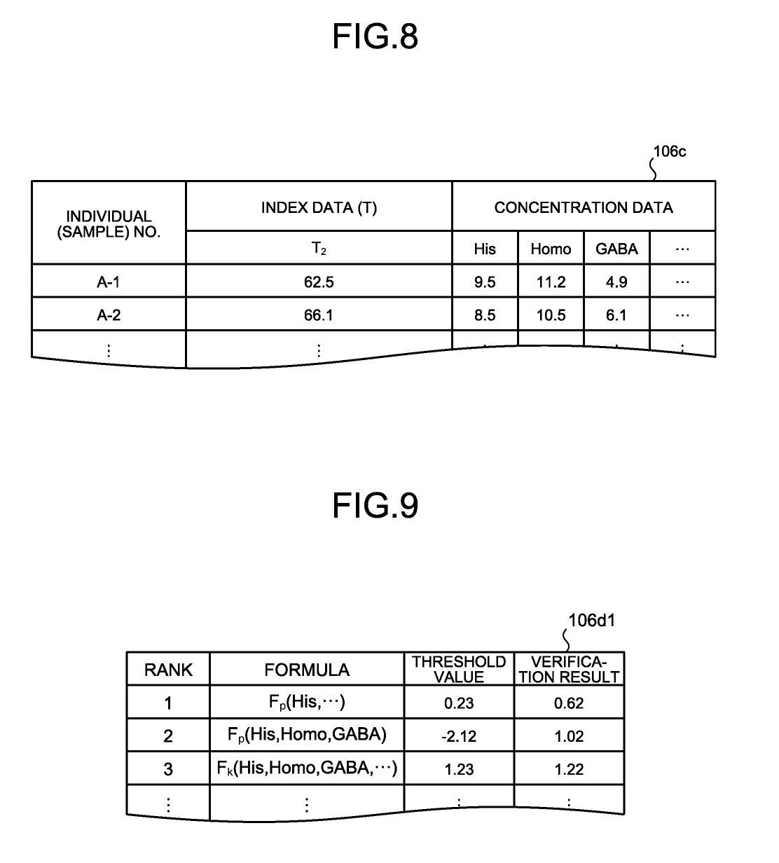

Returning to FIG. 5, the designated index state information file 106c stores the index state information designated in a designating part 102b described below. FIG. 8 is a chart showing an example of information stored in the designated index state information file 106c. As shown in FIG. 8, the information stored in the designated index state information file 106c includes the individual number, the designated index data, and the designated concentration data that are correlated to one another.

Returning to FIG. 5, the formula-related information database 106d is composed of the formula file 106d1 storing the formula prepared in a formula-preparing part 102c described below. The formula file 106d1 stores the formulae used for the evaluation. FIG. 9 is a chart showing an example of information stored in the formula file 106d1. As shown in FIG. 9, the information stored in the formula file 106d1 includes a rank, the formula (e.g., F, (Homo, . . . ), Fₚ (Homo, GABA, Asn), Fₖ (Homo, GABA, Asn, . . . ) in FIG. 9), a threshold corresponding to each formula-preparing method, and the verification result of each formula (e.g., the value of each formula) that are correlated to one another. The character referred to as “Homo” means homoarginine.

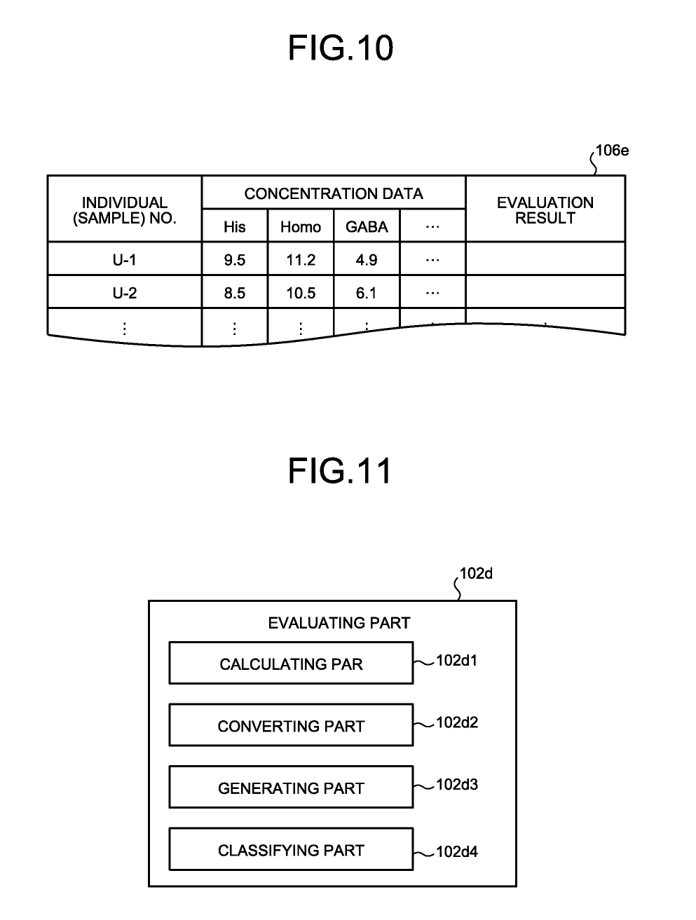

Returning to FIG. 5, the evaluation result file 106e stores the evaluation results obtained in the evaluating part 102d described below. FIG. 10 is a chart showing an example of information stored in the evaluation result file 106e. The information stored in the evaluation result file 106e includes the individual number for uniquely identifying the individual (sample) as the subject, the previously obtained concentration data of the individual, and the evaluation result on the state of colorectal cancer (for example, the value of the formula calculated by a calculating part 102d1 described below, the converted value of the value of the formula obtained by a converting part 102d2 described below, the positional information generated by a generating part 102d3 described below, or the classification result obtained by a classifying part 102d4 described below), that are correlated to one another.

Returning to FIG. 5, the control device 102 has an internal memory storing, for example, control programs such as OS (Operating System), programs for various processing procedures, and other needed data, and performs various information processings according to these programs. As shown in the figure, the control device 102 includes mainly a receiving part 102a, the designating part 102b, the formula-preparing part 102c, the evaluating part 102d, a result outputting part 102e, and a sending part 102f. The control device 102 performs data processings such as removal of data including defective, removal of data including many outliers, and removal of explanatory variables for the defective-including data in the index state information transmitted from the database apparatus 400 and in the concentration data transmitted from the client apparatus 200.

The receiving part 102a receives, via the network 300, information (specifically, the concentration data, the index state information, the formula, etc.) transmitted from the client apparatus 200 and the database apparatus 400. The designating part 102b designates objective index data and objective concentration data in preparing the formula.

The formula-preparing part 102c generates the formula based on the index state information received in the receiving part 102a or the index state information designated in the designating part 102b. If the formulae are stored previously in a predetermined region of the memory device 106, the formula-preparing part 102c may generate the formula by selecting the desired formula out of the memory device 106. Alternatively, the formula-preparing part 102c may generate the formula by selecting and downloading the desired formula from another computer apparatus (e.g., the database apparatus 400) in which the formulae are previously stored.

The evaluating part 102d evaluates the state of colorectal cancer for the individual by calculating the value of the formula using the previously obtained formula (for example, the formula prepared by the formula-preparing part 102c or the formula received by the receiving part 102a) and the concentration value of at least one of the 25 kinds of metabolites and the 19 kinds of amino acids included in the concentration data of the individual received by the receiving part 102a. The evaluating part 102d may evaluate the state of colorectal cancer for the individual using the concentration value of at least one of the 25 kinds of metabolites and the 19 kinds of amino acids or the converted value of the concentration value (for example, the concentration standard score).

Hereinafter, a configuration of the evaluating part 102d will be described with reference to FIG. 11. FIG. 11 is a block diagram showing the configuration of the evaluating part 102d, and only a part in the configuration related to the present invention is shown conceptually. The evaluating part 102d includes the calculating part 102d1, the converting part 102d2, the generating part 102d3, and the classifying part 102d4, additionally.

The calculating part 102d1 calculates the value of the formula using the concentration value of at least one of the 25 kinds of metabolites and the 19 kinds of amino acids and the formula including the explanatory variable to be substituted with the concentration value of at least one of the 25 kinds of metabolites and the 19 kinds of amino acids. The evaluating part 102d may store the value of the formula calculated by the calculating part 102d1 as the evaluation result in a predetermined region of the evaluation result file 106e.

The converting part 102d2 converts the value of the formula calculated by the calculating part 102d1, for example, by the conversion method described above. The evaluating part 102d may store the converted value by the converting part 102d2 as the evaluation result in a predetermined region of the evaluation result file 106e. The converting part 102d2 may convert the concentration value of at least one of the 25 kinds of metabolites and the 19 kinds of amino acids included in the concentration data, for example, by the conversion method described above.

The generating part 102d3 generates the positional information about the position of the predetermined mark on the predetermined scale visually presented on the display device such as a monitor or the physical medium such as paper, using the value of the formula calculated by the calculating part 102d1 or the converted value by the converting part 102d2 (the concentration value or the converted value of the concentration value may be used as well). The evaluating part 102d may store the positional information generated by the generating part 102d3 as the evaluation result in a predetermined region of the evaluation result file 106e.

The classifying part 102d4 classifies the individual into any one of the categories defined at least considering the degree of the possibility of having colorectal cancer, using the value of the formula calculated by the calculating part 102d1 or the converted value by the converting part 102d2 (the concentration value or the converted value of the concentration value may be used as well).

The result outputting part 102e outputs, into the output device 114, for example, the processing results in each processing part in the control device 102 (including the evaluation results obtained by the evaluating part 102d).

The sending part 102f transmits the evaluation results to the client apparatus 200 that is a sender of the concentration data of the individual, and transmits the evaluation formulae prepared in the evaluating apparatus 100 and the evaluation results to the database apparatus 400.

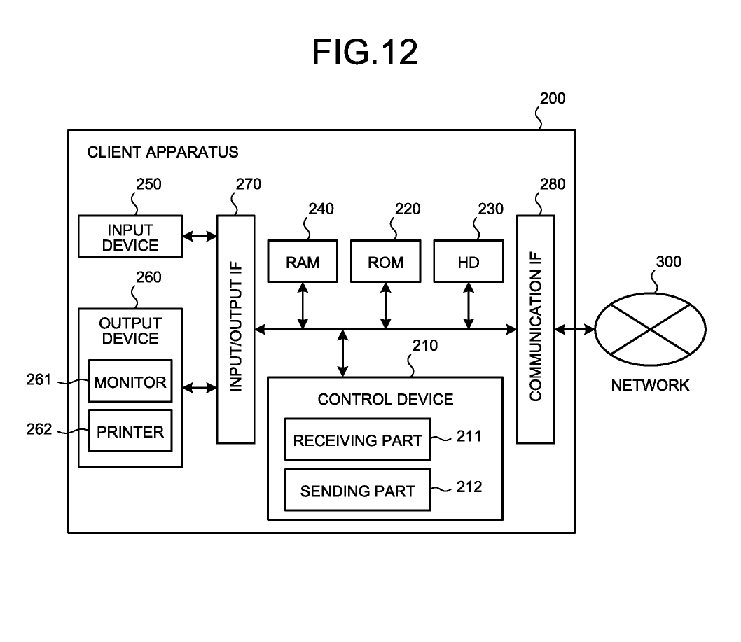

Hereinafter, a configuration of the client apparatus 200 in the present system will be described with reference to FIG. 12. FIG. 12 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel. The client apparatus 200 may be realized based on an information processing apparatus (for example, an information processing terminal such as a known personal computer, a workstation, a family computer, Internet TV (Television), PHS (Personal Handyphone System) terminal, a mobile phone terminal, a mobile unit communication terminal, or PDA (Personal Digital Assistants)) connected as needed with peripheral devices such as a printer, a monitor, and an image scanner.

The input device 250 is, for example, a keyboard, a mouse, or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatus 200 is connected to the network 300 via a communication apparatus such as a modem, TA (Terminal Adapter) or a router, and a telephone line, or via a private line. In this way, the client apparatus 200 can access to the evaluating apparatus 100 by using a particular protocol.

The control device 210 has a receiving part 211 and a sending part 212. The receiving part 211 receives various kinds of information such as the evaluation results transmitted from the evaluating apparatus 100, via the communication IF 280. The sending part 212 sends various kinds of information such as the concentration data of the individual, via the communication IF 280, to the evaluating apparatus 100.

All or a part of processings of the control device 210 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

The control device 210 may include an evaluating part 210a (including a calculating part 210a1, a converting part 210a2, a generating part 210a3, and a classifying part 210a4) having the same functions as the functions of the evaluating part 102d in the evaluating apparatus 100. When the control device 210 includes the evaluating part 210a, the evaluating part 210a may convert the value of the formula (the concentration value may be used as well) in the converting part 210a2, generate the positional information corresponding to the value of the formula or the converted value (the concentration value or the converted value of the concentration value may be used as well) in the generating part 210a3, and classify the individual into any one of the categories using the value of the formula or the converted value (the concentration value or the converted value of the concentration value may be used as well) in the classifying part 210a4, in accordance with information included in the evaluation results transmitted from the evaluating apparatus 100.

Hereinafter, the network 300 in the present system will be described with reference to FIGS. 3 and 4. The network 300 has a function to connect the evaluating apparatus 100, the client apparatuses 200, and the database apparatus 400 mutually, communicatively to one another, and is for example the Internet, an intranet, or LAN (Local Area Network (including both wired and wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (registered trademark) (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

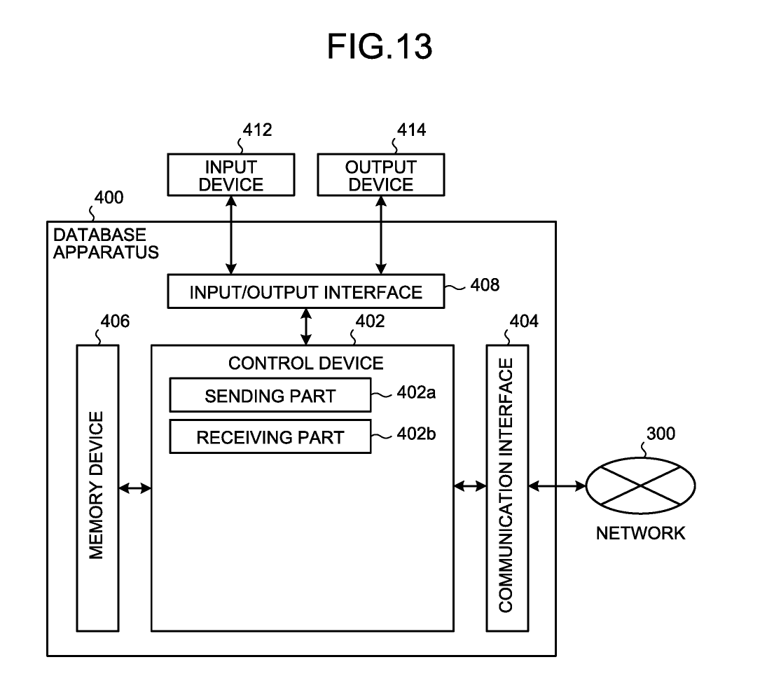

Hereinafter, the configuration of the database apparatus 400 in the present system will be described with reference to FIG. 13. FIG. 13 is a block diagram showing an example of the configuration of the database apparatus 400 in the present system, showing conceptually only the region relevant to the present invention.

The database apparatus 400 has functions to store, for example, the index state information used in preparing the formulae in the evaluating apparatus 100 or the database apparatus, the formulae prepared in the evaluating apparatus 100, and the evaluation results obtained in the evaluating apparatus 100. As shown in FIG. 13, the database apparatus 400 includes (I) a control device 402, such as CPU, that integrally controls the database apparatus, (II) a communication interface 404 connecting the database apparatus to the network 300 communicatively via communication apparatuses such as a router and wired or wireless communication circuits such as a private line, (III) a memory device 406 storing various databases, tables, files (for example, files for Web pages) and others, and (IV) an input/output interface 408 connected to an input device 412 and an output device 414, and these parts are connected communicatively to each other via any communication channel.

The memory device 406 is a storage means, and, examples thereof include a memory apparatus such as RAM or ROM, a fixed disk drive such as a hard disk, a flexible disk, and an optical disk. The memory device 406 stores, for example, various programs used in various processings. The communication interface 404 allows communication between the database apparatus 400 and the network 300 (or a communication apparatus such as a router). Thus, the communication interface 404 has a function to communicate data via a communication line with other terminals. The input/output interface 408 is connected to the input device 412 and the output device 414. A monitor (including a home television), a speaker, or a printer may be used as the output device 414. A keyboard, a mouse, a microphone, or a monitor functioning as a pointing device together with a mouse may be used as the input device 412.

The control device 402 has an internal memory storing, for example, control programs such as OS (Operating System), programs for various processing procedures, and other needed data, and performs various information processings according to these programs. As shown in the figure, the control device 402 includes mainly a sending part 402a and a receiving part 402b. The sending part 402a transmits various kinds of information such as the index state information and the formulae to the evaluating apparatus 100. The receiving part 402b receives various kinds of information such as the formula and the evaluation results, transmitted from the evaluating apparatus 100.

In the present description, the evaluating apparatus 100 executes the reception of the concentration data, the calculation of the value of the formula, the classification of the individual into the category, and the transmission of the evaluation results, while the client apparatus 200 executes the reception of the evaluation results, described as an example. However, when the client apparatus 200 includes the evaluating unit 210a, the evaluating apparatus 100 only has to execute the calculation of the value of the formula. For example, the conversion of the value of the formula, the generation of the positional information, and the classification of the individual into the category may be appropriately shared between the evaluating apparatus 100 and the client apparatus 200.

For example, when the client apparatus 200 receives the value of the formula from the evaluating apparatus 100, the evaluating unit 210a may convert the value of the formula in the converting unit 210a2, generate the positional information corresponding to the value of the formula or the converted value in the generating unit 210a3, and classify the individual into any one of the categories using the value of the formula or the converted value in the classifying unit 210a4.

When the client apparatus 200 receives the converted value from the evaluating apparatus 100, the evaluating unit 210a may generate the positional information corresponding to the converted value in the generating unit 210a3, and classify the individual into any one of the categories using the converted value in the classifying unit 210a4.

When the client apparatus 200 receives the value of the formula or the converted value and the positional information from the evaluating apparatus 100, the evaluating unit 210a may classify the individual into any one of the categories using the value of the formula or the converted value in the classifying unit 210a4.

### 2-3. Other Embodiments

In addition to the second embodiment described above, the evaluating apparatus, the evaluating method, the evaluating program product, the evaluating system, and the terminal apparatus according to the present invention can be practiced in various different embodiments within the technological scope of the claims.

Of the processings described in the second embodiment, all or a part of the processings described as automatically performed ones may be manually performed, or all or a part of the processings described as manually performed ones may be also automatically performed by known methods.

In addition, the processing procedures, the control procedures, the specific names, the information including parameters such as registered data of various processings and retrieval conditions, the screen examples, and the database configuration shown in the description and the drawings may be arbitrarily modified unless otherwise specified.

The components of the evaluating apparatus 100 shown in the figures are functionally conceptual and therefore not be physically configured as shown in the figures.

For example, for the operational functions provided in the evaluating apparatus 100, in particular, for the operational functions performed in the control device 102, all or part thereof may be implemented by the CPU (Central Processing Unit) and programs interpreted and executed in the CPU, or may be implemented by wired-logic hardware. The program is recorded in a non-transitory tangible computer-readable recording medium including programmed instructions for making an information processing apparatus execute the evaluating method according to the present invention, and is mechanically read as needed by the evaluating apparatus 100. More specifically, computer programs to give instructions to the CPU in cooperation with the OS (operating system) to perform various processes are recorded in the memory device 106 such as ROM or a HDD (hard disk drive). The computer programs are executed by being loaded to RAM, and form the control unit in cooperation with the CPU.

The computer programs may be stored in an application program server connected to the evaluating apparatus 100 via an arbitrary network, and all or part thereof can be downloaded as necessary.

The evaluating program according to the present invention may be stored in the non-transitory tangible computer-readable recording medium, or can be configured as a program product. The “recording medium” mentioned here includes any “portable physical medium” such as a memory card, a USB (universal serial bus) memory, an SD (secure digital) card, a flexible disk, a magneto-optical disc, ROM, EPROM (erasable programmable read only memory), EEPROM (registered trademark) (electronically erasable and programmable read only memory), CD-ROM (compact disk read only memory), MO (magneto-optical disk), DVD (digital versatile disk), and Blu-ray (registered trademark) Disc.

The “program” mentioned here is a data processing method described in an arbitrary language or description method, and therefore any form such as a source code and a binary code is acceptable. The “program” is not necessarily limited to a program configured as a single unit, and, therefore, includes those dispersively configured as a plurality of modules and libraries and those in which the function of the program is achieved in cooperation with separate programs represented as OS (operating system). Any known configuration and procedures can be used as a specific configuration and reading procedure to read a recording medium by each apparatus shown in the embodiments, an installation procedure after the reading, and the like.

The various databases and the like stored in the memory device 106 is a storage unit such as a memory device such as RAM and ROM, a fixed disk drive such as a hard disk, a flexible disk, or an optical disc. The memory device 106 stores therein various programs, tables, databases, files for Web (World Wide Web) pages, and the like used to perform various processes and to provide Web sites.

The evaluating apparatus 100 may be configured as an information processing apparatus such as known personal computer and work station, or may be configured as the information processing apparatus connected to an arbitrary peripheral device. The evaluating apparatus 100 may be provided by installing software (including the programs and the data, etc.) to cause the information processing apparatus to implement the evaluating method according to the present invention.

Furthermore, a specific configuration of dispersion or integration of the apparatuses is not limited to the shown one. The apparatuses can be configured by functionally or physically dispersing or integrating all or part of the apparatuses in arbitrary units according to various types of additions or the like or according to functional loads. In other words, the embodiments may be implemented in arbitrary combinations thereof or an embodiment may be selectively implemented.

### Example 1

The plasma samples of colorectal cancer patients (colorectal cancer group: 40 people) who were given a definitive diagnosis of colorectal cancer and the plasma samples of healthy subjects (healthy group: 40 people) who have neither anamnesis of cancer nor morbidity of cancer and are selected to match the colorectal cancer group in gender, age, and BMI were measured to determine blood concentrations of metabolites by the above-mentioned metabolite analyzing method (A).

The discrimination ability of the colorectal cancer group and the healthy group was evaluated for each metabolite based on the ROC_AUC (area under the curve in the receiver operating characteristic curve) with the data on the plasma concentration values (nmol/ml) of 22 metabolites (1-Me-His, aABA, Aminoadipic acid, bABA, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, and SDMA). Table 1 indicates ROC AUCs serving as indexes to evaluate the discrimination ability of the respective metabolites.

TABLE 1

| | | ASYMPTOTIC |
|---|---|---|
| | ROC_AUC | P-VALUE |
| 1-Me-His | 0.4025 | 0.5423 |
| aABA | 0.6331 | 0.0346 |
| Aminoadipic acid | 0.6347 | 0.0481 |
| bABA | 0.9497 | <0.0001 |
| bAiBA | 0.5744 | 0.1567 |
| Cadaverine | 0.7806 | 0.0004 |
| Ethylglycine | 0.6228 | 0.6749 |
| GABA | 0.6472 | 0.1093 |
| Homoarginine | 0.5859 | 0.1876 |
| Hypotaurine | 0.5391 | 0.2751 |
| Kinurenine | 0.5172 | 0.2875 |
| N6-Acetyl-L-Lys | 0.5531 | 0.9449 |
| Putrescine | 0.5819 | 0.3141 |
| Serotonin | 0.4809 | 0.0877 |
| Spermidine | 0.5381 | 0.4781 |
| Spermine | 0.6288 | 0.0197 |
| ADMA | 0.6600 | 0.0131 |
| Homocitrulline | 0.4856 | 0.3969 |
| 3-Me-His | 0.4191 | 0.8897 |
| Hydroxyproline | 0.4253 | 0.4807 |
| Phosphoethanolamine | 0.5156 | 0.1694 |
| SDMA | 0.6400 | 0.0265 |

In the test with null hypothesis of “ROC_AUC=0.5” under nonparametric assumption, metabolites having significant ROC_AUC (p<0.05) were aABA, Aminoadipic acid, bABA, Cadaverine, Spermine, ADMA, and SDMA. In the colorectal cancer group, aABA, Aminoadipic acid, and ADMA significantly decreased, but bABA, Cadaverine, Spermine, and SDMA significantly increased. Because the ROC AUCs were significant, the concentration values of those metabolites were considered to be valuable in the evaluation of the state of colorectal cancer which takes into account the healthy states.

### Example 2

The sampled data obtained in Example 1 were used. A multivariate discriminant (multivariate function) that determines two groups of the colorectal cancer group and the healthy group was obtained, the multivariate discriminant including explanatory variables to be substituted with plasma concentration values of metabolites.

The logistic regression equation was used as the multivariate discriminant. The combinations of two explanatory variables to be included in a logistic regression equation were searched on 19 amino acids (Asn, His, Thr, Ala, Cit, Arg, Tyr, Val, Met, Lys, Trp, Gly, Pro, Orn, Ile, Leu, Phe, Ser, and Gln) and the above-mentioned 22 metabolites under the condition that at least one of the 22 metabolites is inevitably included. Next, logistic regression equations with excellent discrimination ability discriminating the colorectal cancer group and the healthy group were searched.

The logistic regression equations each including two explanatory variables and making the ROC_AUC values for the colorectal cancer group and the healthy group equal to 0.700 or more are listed in [11. Formula with two variables] detailed later. These logistic regression equations having high ROC_AUC values are considered to be valuable in the above-discussed evaluation. In [11. Formula with two variables], explanatory variables included in formula and an ROC_AUC value for each formula are shown (the same applies hereafter).

### Example 3

The sample data used in Example 1 is used. A multivariate discriminant (multivariate function) that determines two groups of the colorectal cancer group and the healthy group was obtained, the multivariate discriminant including explanatory variables to be substituted with plasma concentration values of metabolites.

The logistic regression equation was used as the multivariate discriminant. The combinations of three explanatory variables to be included in a logistic regression equation were searched on the above-mentioned 19 amino acids and the above-mentioned 22 metabolites under the condition that, similarly to Example 2, at least one of the 22 metabolites is inevitably included. Next, logistic regression equations with excellent discrimination ability discriminating the colorectal cancer group and the healthy group were searched.

The logistic regression equations each including three explanatory variables and making the ROC_AUC values for the colorectal cancer group and the healthy group equal to 0.955 (the maximum ROC_AUC value in Example 2) or more are listed in [12. Formula with three variables] detailed later. These logistic regression equations having high ROC_AUC values are considered to be valuable in the above-discussed evaluation.

### Example 4

The sample data used in Example 1 is used. A multivariate discriminant (multivariate function) that determines two groups of the colorectal cancer group and the healthy group was obtained, the multivariate discriminant including explanatory variables to be substituted with plasma concentration values of metabolites.

The logistic regression equation was used as the multivariate discriminant. The combinations of six explanatory variables to be included in a logistic regression equation were searched on the above-mentioned 19 amino acids and the above-mentioned 22 metabolites. Next, logistic regression equations with excellent discrimination ability discriminating the colorectal cancer group and the healthy group were searched.

Out of the obtained logistic regression equations, the 36 expressions the ROC_AUC of which is 0.980 (the maximum ROC_AUC value in Example 3) or more were examined to determine the appearance frequencies of explanatory variables of amino acids included therein. The logistic regression equations are listed in [13. Formula with six variables] detailed later, and the appearance frequencies are shown in Table 2. The result shows that Ser, Val, Orn, Leu, Trp, bABA, Cadaverine, Serotonin, and ADMA have high appearance frequencies equal to 10 or more. In particular, Val, Leu, bABA, and Cadaverine have higher appearance frequencies equal to 20 or more. Furthermore, Val, bABA, and Cadaverine have higher appearance frequencies equal to 30 or more.

TABLE 2

| | ROC_AUC |
|---|---|
| | EQUAL TO OR |
| | MORE THAN 0.980 |
| Thr | 0 |
| Ser | 12 |
| Asn | 0 |
| Gln | 0 |
| Pro | 4 |
| Gly | 0 |
| Ala | 0 |
| Cit | 3 |
| Val | 36 |
| Met | 0 |
| Ile | 0 |
| Leu | 22 |
| Tyr | 0 |
| Phe | 0 |
| His | 5 |
| Trp | 11 |
| Orn | 16 |
| Lys | 0 |
| Arg | 0 |
| 1-Me-His | 0 |
| aABA | 4 |
| Aminoadipic acid | 5 |
| bABA | 36 |
| bAiBA | 0 |
| Cadaverine | 36 |
| Ethylglycine | 0 |
| GABA | 0 |
| Homoarginine | 0 |
| Hypotaurine | 0 |
| Kinurenine | 0 |
| N6-Acetyl-L-Lys | 0 |
| Putrescine | 0 |
| Serotonin | 12 |
| Spermidine | 0 |
| Spermine | 7 |
| ADMA | 14 |
| Homocitrulline | 0 |
| 3-Me-His | 0 |
| Hydroxyproline | 0 |
| Phosphoethanolamine | 0 |
| SDMA | 5 |

Among the obtained logistic regression equations, for example, the discrimination ability of Index Formula 1 having the combination of explanatory variables “Val, Leu, bABA, Cadaverine, Serotonin, and SDMA” (multivariate discriminant including Val, Leu, bABA, Cadaverine, Serotonin, and SDMA as explanatory variables) was excellent, specifically, ROC_AUC=0.9844, sensitivity=0.950, and specificity=0.950. Note that the values of the sensitivity and the specificity were determined when the cutoff value was set to the highest discriminant point that produces the highest average of the sensitivity and the specificity.

The values of the expressions were calculated based on Index Formula 1 and the concentration values (4 mol/L) of the amino acids and the metabolites of the colorectal cancer group. Next, each of the cases on the colorectal cancer group was sorted into one of a plurality of categories based on the derived values of the expressions and the preset cutoff values, the categories being determined as shown later. As the candidates of the cutoff values, the value of the expression when the specificity is 80% and the value of the expression when the specificity is 95% were calculated, and were −1.914 and −0.034, respectively. Note that, when the cutoff values were set to the candidate values, the respective sensitivities were 100% and 95%.

The concentration values of the case having the highest value of expression were Val: 132.1, Leu: 86.0, bABA: 0.411, Cadaverine: 0.0266, Serotonin: 0.246, and SDMA: 0.627. The value of expression of the case was 16.7. Logarithmic odds ln(p/(1−p))=a value of expression was defined as a relational expression (where p is a probability of cancer). Then, the odds p/(1−p) were calculated based on the value of expression that was 16.7. The determined odds were 17894429.1. In addition, the determined probability p based on the odds was 1.0.

Next, the cutoff value was set to −0.034 that was the value of expression when the specificity was 95%. A value of expression higher than the cutoff value was defined as positive (corresponding to the colorectal cancer category) and lower than the cutoff value was defined as negative (corresponding to the healthy category). Sorting of the case having the value of expression equal to 16.7 into either the positive or the negative was attempted. This case was sorted into the positive because the value of expression was higher than the cutoff value.

In addition, a first cutoff value was defined and set to the value of expression that was −1.914 when the specificity was 80%, and a second cutoff value was defined and set to the value of expression that was −0.034 when the specificity was 95%. A value of expression lower than the first cutoff value was defined as Rank A (category where the possibility (probability or risk) of colorectal cancer is low), a value of expression higher than the first cutoff value and lower than the second cutoff value was defined as Rank B (category where the possibility of colorectal cancer is middle), and a value of expression higher than the second cutoff value was defined as Rank C (category where the possibility of colorectal cancer is high). By sorting the case having the value of expression equal to 16.7 into one of the three ranks, this case was sorted into Rank C because the value of expression was higher than the second cutoff value.

### Example 5

In Example 5, in addition to blood concentrations of the metabolites in Example 1, peak area values of N-methyl-β-aminobutyric acid (N-Me-bABA), Acylcarnitine (AC) (13:1), and cis-5,8,11,14,17-Eicosapentaenoic acid (EPA) in blood were measured by the above-mentioned metabolite analyzing method (A) using plasma of 24 healthy subjects and plasma of 24 colorectal cancer patients being selected from the blood samples used in Example 1. The measured peak area values are treated as concentration values.

The discrimination ability of the colorectal cancer group and the healthy group was evaluated for each metabolite based on the ROC_AUC with the data on the concentration values (peak area values) of 3 metabolites (N-Me-bABA, AC (13:1), and EPA). Table 3 indicates ROC AUCs serving as indexes to evaluate the discrimination ability of the respective metabolites.

TABLE 3

| | | ASYMPTOTIC |
|---|---|---|
| | ROC_AUC | P-VALUE |
| N-Me-bABA | 0.9323 | <0.0001 |
| AC (13:1) | 0.8472 | <0.0001 |
| EPA | 0.7813 | 0.0007 |

In the test with null hypothesis of “ROC_AUC=0.5” under nonparametric assumption, N-Me-bABA, AC (13:1), and EPA had significant ROC_AUC (p<0.05), and furthermore, in the colorectal cancer group, AC (13:1) and EPA significantly decreased, and N-Me-bABA significantly increased. Because the ROC_AUC was significant, the concentration values of these metabolides were considered to be valuable in the evaluation of the state of colorectal cancer which takes into account the healthy states.

### Example 6

The sampled data obtained in Example 5 were used. A multivariate discriminant (multivariate function) that determines two groups of the colorectal cancer group and the healthy group was obtained, the multivariate discriminant including explanatory variables to be substituted with plasma concentration values of metabolites.

The logistic regression equation was used as the multivariate discriminant. The combinations of two explanatory variables to be included in a logistic regression equation were searched on the above-mentioned 19 amino acids and the above-mentioned 22 metabolites under the condition that N-Me-bABA, AC (13:1), or EPA is inevitably included. Next, logistic regression equations with excellent discrimination ability discriminating the colorectal cancer group and the healthy group were searched.

The logistic regression equations (combination of explanatory variables) each including two explanatory variables and making the ROC_AUC values for the colorectal cancer group and the healthy group equal to 0.9323 (the maximum ROC_AUC value in Example 5) or more are listed in [14. Formula with two variables] detailed later. These logistic regression equations having high ROC_AUC values are considered to be valuable in the above-discussed evaluation.

### Example 7

The sample data used in Example 5 is used. A multivariate discriminant (multivariate function) that determines two groups of the colorectal cancer group and the healthy group was obtained, the multivariate discriminant including explanatory variables to be substituted with plasma concentration values of metabolites or peak area values.

The logistic regression equation was used as the multivariate discriminant. An explanatory variable or a combination of two explanatory variables to be added in the logistic regression equation having six amino acids of Ser, Pro, Val, Met, Ile, and Lys as the explanatory variables and making the ROC_AUC value for the colorectal cancer group and the healthy group equal to 0.9132 were searched on the above-mentioned 22 metabolites and the above-mentioned 3 metabolites. Next, logistic regression equations with excellent discrimination ability discriminating the colorectal cancer group and the healthy group were searched.

In the search when an explanarory variable is added, the metabolites added in the logistic regression equations each making the ROC_AUC values for the colorectal cancer group and the healthy group equal to 0.9132 or more were listed in [15: One variable to be added] detailed later. In the search when two explanarory variables are added, the metabolites added in the logistic regression equations each making the ROC_AUC values for the colorectal cancer group and the healthy group equal to 0.9132 or more were listed in [16: Two variables to be added] detailed later. These logistic regression equations having high ROC_AUC values are considered to be valuable in the above-discussed evaluation.

[11. Formula with Two Variables]

Orn, bABA, 0.9550; Gly, bABA, 0.9544; Ile, bABA, 0.9538; Asn, bABA, 0.9519; Ala, bABA, 0.9519; Pro, bABA, 0.9519; Trp, bABA, 0.9513; Thr, bABA, 0.9506; Tyr, bABA, 0.9506; Phe, bABA, 0.9500; Gln, bABA, 0.9500; Arg, bABA, 0.9500; Lys, bABA, 0.9500; Val, bABA, 0.9494; Met, bABA, 0.9494; Ser, bABA, 0.9488; Leu, bABA, 0.9488; Cit, bABA, 0.9481; His, bABA, 0.9475; Trp, Cadaverine, 0.8681; Val, Cadaverine, 0.8519; Orn, Cadaverine, 0.8425; Leu, Cadaverine, 0.8231; Trp, Spermine, 0.8144; His, Cadaverine, 0.8081; Cit, Cadaverine, 0.8075; Gly, Cadaverine, 0.8044; Lys, Cadaverine, 0.8013; Met, Cadaverine, 0.7988; Trp, ADMA, 0.7963; Pro, Cadaverine, 0.7925; Ala, Cadaverine, 0.7919; Arg, Cadaverine, 0.7875; Tyr, Cadaverine, 0.7863; Phe, Cadaverine, 0.7856; Orn, Aminoadipic acid, 0.7831; Gln, Cadaverine, 0.7819; Trp, SDMA, 0.7813; Trp, GABA, 0.7794; Ile, Cadaverine, 0.7788; Asn, Cadaverine, 0.7788; Ser, Cadaverine, 0.7775; Trp, bAiBA, 0.7756; Trp, Putrescine, 0.7738; Trp, Phosphoethanolamine, 0.7738; Trp, Kynurenine, 0.7731; Trp, Hypotaurine, 0.7731; Trp, 3-Me-His, 0.7731; Thr, Cadaverine, 0.7719; Trp, Homoarginine, 0.7706; Trp, Ethylglycine, 0.7700; Trp, Serotonin, 0.7694; Trp, HyPro, 0.7694; Trp, aABA, 0.7688; Trp, Homocitrulline, 0.7688; Trp, Spermidine, 0.7675; Trp, 3-Me-His, 0.7663; Trp, N6-Acetyl-L-Lys, 0.7663; Trp, Aminoadipic acid, 0.7631; His, ADMA, 0.7575; His, SDMA, 0.7569; Val, Spermine, 0.7556; Val, ADMA, 0.7494; Val, GABA, 0.7469; His, Spermine, 0.7450; Val, SDMA, 0.7450; His, GABA, 0.7444; Orn, GABA, 0.7406; Cit, GABA, 0.7375; His, bAiBA, 0.7300; Pro, Aminoadipic acid, 0.7288; His, Serotonin, 0.7256; Val, bAiBA, 0.7238; Orn, Spermine, 0.7225; His, 3-Me-His, 0.7219; His, Aminoadipic acid, 0.7213; His, Kynurenine, 0.7200; Orn, aABA, 0.7194; Val, Kynurenine, 0.7169; His, Homocitrulline, 0.7163; His, Putrescine, 0.7150; Orn, SDMA, 0.7150; His, Phosphoethanolamine, 0.7144; Leu, ADMA, 0.7144; Orn, Putrescine, 0.7138; Val, Serotonin, 0.7131; His, aABA, 0.7119; His, Ethylglycine, 0.7106; His, Homoarginine, 0.7100; Val, HyPro, 0.7100; Val, PButrescine, 0.7100; His, Spermidine, 0.7088; Leu, Spermine, 0.7088; His, Hypotaurine, 0.7081; Met, ADMA, 0.7081; Orn, ADMA, 0.7075; Val, Homocitrulline, 0.7069; Val, Ethylglycine, 0.7063; Val, Hypotaurine, 0.7056; Val, aABA, 0.7044; Val, Homoarginine, 0.7044; Orn, N6-Acetyl-L-Lys, 0.7044; His, 3-Me-His, 0.7044; Val, 3-Me-His, 0.7038; Orn, Spermidine, 0.7031; Pro, aABA, 0.7025; Val, Aminoadipic acid, 0.7019; Val, Phosphoethanolamine, 0.7012; Orn, 3-Me-His, 0.7006; Val, Spermidine, 0.7000

[12. Formula with Three Variables]

Gly, bABA, Cadaverine, 0.9800; Orn, bABA, Cadaverine, 0.9725; Val, bABA, Cadaverine, 0.9663; Ile, 3-Me-His, bABA, 0.9656; Gly, 3-Me-His, bABA, 0.9650; Ala, bABA, Cadaverine, 0.9650; Trp, bABA, Cadaverine, 0.9644; Ile, bABA, Cadaverine, 0.9644; Gly, bABA, ADMA, 0.9638; Arg, bABA, Cadaverine, 0.9638; Leu, bABA, Cadaverine, 0.9638; Gln, bABA, Cadaverine, 0.9638; His, bABA, Cadaverine, 0.9631; Phe, bABA, Cadaverine, 0.9631; Cit, bABA, Cadaverine, 0.9625; Orn, bABA, GABA, 0.9625; Ser, bABA, Cadaverine, 0.9619; His, 3-Me-His, bABA, 0.9619; Tyr, bABA, Cadaverine, 0.9619; Met, bABA, Cadaverine, 0.9619; Lys, bABA, Cadaverine, 0.9619; Trp, 3-Me-His, bABA, 0.9619; Val, bABA, GABA, 0.9613; Thr, bABA, Cadaverine, 0.9613; Pro, bABA, Cadaverine, 0.9613; Thr, 3-Me-His, bABA, 0.9613; Pro, 3-Me-His, bABA, 0.9613; Tyr, 3-Me-His, bABA, 0.9613; Cit, bABA, GABA, 0.9606; Ile, bABA, ADMA, 0.9600; Val, 3-Me-His, bABA, 0.9600; Orn, 3-Me-His, bABA, 0.9600; Asn, bABA, Cadaverine, 0.9594; Gly, bABA, N6-Acetyl-L-Lys, 0.9594; Gln, 3-Me-His, bABA, 0.9594; Cit, 3-Me-His, bABA, 0.9594; Arg, 3-Me-His, bABA, 0.9594; Orn, aABA, bABA, 0.9594; Phe, 3-Me-His, bABA, 0.9594; Ser, 3-Me-His, bABA, 0.9594; Thr, bABA, Serotonin, 0.9588; Orn, bABA, HyPro, 0.9588; Ile, bABA, Spermine, 0.9588; Asn, 3-Me-His, bABA, 0.9588; Ala, 3-Me-His, bABA, 0.9588; Met, 3-Me-His, bABA, 0.9588; Asn, bABA, GABA, 0.9581; Gly, bABA, Spermine, 0.9581; Gln, bABA, Spermine, 0.9581; Ile, bABA, Serotonin, 0.9581; Trp, bABA, Spermine, 0.9581; Leu, 3-Me-His, bABA, 0.9581; Asn, bABA, Spermine, 0.9575; Gly, Aminoadipic acid, bABA, 0.9575; Thr, bABA, Spermine, 0.9575; Val, bABA, Spermine, 0.9575; Gly, bABA, bAiBA, 0.9575; Val, bABA, Homoarginine, 0.9575; Lys, 3-Me-His, bABA, 0.9575; Ile, aABA, bABA, 0.9575; Ile, bABA, bAiBA, 0.9575; Ile, bABA, N6-Acetyl-L-Lys, 0.9575; Ile, bABA, Spermidine, 0.9575; Gly, bABA, GABA, 0.9569; Pro, bABA, Serotonin, 0.9569; Orn, bABA, Homoarginine, 0.9569; Orn, bABA, 3-Me-His, 0.9569; Ile, Aminoadipic acid, bABA, 0.9569; Trp, bABA, ADMA, 0.9569; Ile, bABA, SDMA, 0.9569; Orn, bABA, Homocitrulline, 0.9563; Gly, bABA, Ethylglycine, 0.9563; Gly, bABA, SDMA, 0.9563; Tyr, bABA, Spermine, 0.9563; Val, bABA, SDMA, 0.9563; Orn, bABA, bAiBA, 0.9563; Orn, bABA, Kynurenine, 0.9563; Orn, bABA, Spermine, 0.9563; Orn, bABA, Phosphoethanolamine, 0.9563; Orn, bABA, SDMA, 0.9563; His, bABA, SDMA, 0.9556; Val, bABA, bAiBA, 0.9556; Val, bABA, N6-Acetyl-L-Lys, 0.9556; Met, bABA, Spermine, 0.9556; Orn, Aminoadipic acid, bABA, 0.9556; Orn, bABA, Spermidine, 0.9556; Ile, bABA, GABA, 0.9556; Leu, bABA, Spermine, 0.9556; Gly, bABA, Homocitrulline, 0.9556; Gly, bABA, Phosphoethanolamine, 0.9556; Pro, bABA, Spermine, 0.9556; Phe, bABA, Spermine, 0.9556; Gly, bABA, Putrescine, 0.9556; Asn, bABA, Serotonin, 0.9550; Trp, bABA, GABA, 0.9550

[13. Formula with Six Variables]

Val, Orn, Leu, bABA, Cadaverine, ADMA, 0.9888; Val, Orn, bABA, Cadaverine, Serotonin, ADMA, 0.9863; Val, Orn, bABA, Cadaverine, Spermine, ADMA, 0.9850; Val, Leu, bABA, Cadaverine, Serotonin, SDMA, 0.9844; Val, Orn, Aminoadipic acid, bABA, Cadaverine, ADMA, 0.9844; Val, Leu, Trp, bABA, Cadaverine, Spermine, 0.9838; Val, Leu, Trp, bABA, Cadaverine, Serotonin, 0.9838; Val, Leu, Aminoadipic acid, bABA, Cadaverine, Serotonin, 0.9831; Val, Orn, Leu, Trp, bABA, Cadaverine, 0.9831; Val, Leu, bABA, Cadaverine, Serotonin, ADMA, 0.9825; Val, Leu, Trp, bABA, Cadaverine, ADMA, 0.9825; Pro, Val, Leu, Trp, bABA, Cadaverine, 0.9825; His, Val, Orn, bABA, Cadaverine, ADMA, 0.9825; Cit, Val, Orn, bABA, Cadaverine, ADMA, 0.9819; Val, Aminoadipic acid, bABA, Cadaverine, Serotonin, SDMA, 0.9819; Val, Leu, aABA, bABA, Cadaverine, Serotonin, 0.9819; Val, Leu, Trp, Aminoadipic acid, bABA, Cadaverine, 0.9819; Val, Leu, Trp, aABA, bABA, Cadaverine, 0.9819; Val, Orn, Leu, bABA, Cadaverine, Serotonin, 0.9819; Cit, Val, Leu, bABA, Cadaverine, ADMA, 0.9819; Val, Aminoadipic acid, bABA, Cadaverine, Serotonin, ADMA, 0.9813; Val, Orn, bABA, Cadaverine, ADMA, SDMA, 0.9813; Pro, Val, Orn, bABA, Cadaverine, ADMA, 0.9813; Val, Leu, Trp, bABA, Cadaverine, SDMA, 0.9813; Pro, Val, Leu, bABA, Cadaverine, Serotonin, 0.9813; Cit, Val, Leu, Trp, bABA, Cadaverine, 0.9813; His, Val, Leu, Trp, bABA, Cadaverine, 0.9813; Val, Orn, aABA, bABA, Cadaverine, Spermine, 0.9806; Pro, Val, Orn, bABA, Cadaverine, Spermine, 0.9806; Val, Leu, bABA, Cadaverine, Serotonin, Spermine, 0.9806; His, Val, Leu, bABA, Cadaverine, Serotonin, 0.9806; His, Val, Orn, Leu, bABA, Cadaverine, 0.9806; Val, Leu, bABA, Cadaverine, ADMA, SDMA, 0.9800; Val, Orn, aABA, bABA, Cadaverine, ADMA, 0.9800; Val, Orn, Trp, bABA, Cadaverine, Spermine, 0.9800; His, Val, Orn, bABA, Cadaverine, Spermine, 0.9800

[14. Formula with Two Variables]

His, N-Me-bABA, 0.9375; bABA, EPA, 0.9618; Cadaverine, N-Me-bABA, 0.9566; Hypotaurine, N-Me-bABA, 0.9340; Serotonin, N-Me-bABA, 0.9427; SDMA, N-Me-bABA, 1.0000; Gln, N-Me-bABA, 0.9323; Cit, N-Me-bABA, 0.9444; Ile, N-Me-bABA, 0.9531; 1-Me-His, N-Me-bABA, 0.9444; bAiBA, N-Me-bABA, 0.9410; ADMA, N-Me-bABA, 0.9375; SDMA, AC (13:1), 1.0000; Gly, N-Me-bABA, 0.9566; Ala, N-Me-bABA, 0.9410; Tyr, N-Me-bABA, 0.9340; Trp, N-Me-bABA, 0.9375; bABA, N-Me-bABA, 0.9670; GABA, N-Me-bABA, 0.9375; N6-Acetyl-L-Lys, N-Me-bABA, 0.9340; Spermine, N-Me-bABA, 0.9427; SDMA, EPA, 1.0000; Thr, N-Me-bABA, 0.9358; Pro, N-Me-bABA, 0.9531; Orn, N-Me-bABA, 0.9427; bABA, AC (13:1), 0.9878; Ethylglycine, N-Me-bABA, 0.9358; Kynurenine, N-Me-bABA, 0.9340; Spermidine, N-Me-bABA, 0.9358

[15. One Variable to be Added]

1-Me-His, 0.9167; Aminoadipic acid, 0.9201; bABA, 0.9965; Cadaverine, 0.9583; Ethylglycine, 0.9149; GABA, 0.9410; Homoarginine, 0.9167; Kynurenine, 0.9253; N6-Acetyl-L-Lys, 0.9288; Putrescine, 0.9271; Serotonin, 0.9184; Spermidine, 0.9288; Spermine, 0.9149; ADMA, 0.9219; Homocitrulline, 0.9184; 3-Me-His, 0.9236; Hydroxyproline, 0.9236; Phosphoetanolamine, 0.9149; SDMA, 1.0000; N-Me-bABA, 0.9809; AC (13:1), 0.9531; EPA, 0.9410

[16. Two Variables to be Added]

1-Me-His, Aminoadipic acid, 0.9167; 1-Me-His, bABA, 1.0000; 1-Me-His, bAiBA, 0.9149; 1-Me-His, Cadaverine, 0.9601; 1-Me-His, Ethylglycine, 0.9167; 1-Me-His, GABA, 0.9410; 1-Me-His, Homoarginine, 0.9167; 1-Me-His, Kynurenine, 0.9253; 1-Me-His, N6-Acetyl-L-Lys, 0.9253; 1-Me-His, Putrescine, 0.9271; 1-Me-His, Serotonin, 0.9167; 1-Me-His, Spermidine, 0.9288; 1-Me-His, Spermine, 0.9167; 1-Me-His, ADMA, 0.9236; 1-Me-His, Homocitrulline, 0.9167; 1-Me-His, 3-Me-His, 0.9306; 1-Me-His, Hydroxyproline, 0.9236; 1-Me-His, Phosphoetanolamine, 0.9149; 1-Me-His, SDMA, 1.0000; 1-Me-His, N-Me-bABA, 0.9809; 1-Me-His, AC (13:1), 0.9514; 1-Me-His, EPA, 0.9479; aABA, Amino adipic acid, 0.9219; aABA, bABA, 0.9965; aABA, Cadaverine, 0.9583; aABA, GABA, 0.9392; aABA, Homoarginine, 0.9167; aABA, Hypotaurine, 0.9149; aABA, Kynurenine, 0.9253; aABA, N6-Acetyl-L-Lys, 0.9340; aABA, Putrescine, 0.9271; aABA, Serotonin, 0.9167; aABA, Spermidine, 0.9306; aABA, ADMA, 0.9253; aABA, Homocitrulline, 0.9201; aABA, 3-Me-His, 0.9253; aABA, Hydroxyproline, 0.9236; aABA, SDMA, 1.0000; aABA, N-Me-bABA, 0.9844; aABA, AC (13:1), 0.9566; aABA, EPA, 0.9375; Aminoadipic acid, bABA, 1.0000; Aminoadipic acid, bAiBA, 0.9149; Aminoadipic acid, Cadaverine, 0.9601; Aminoadipic acid, Ethylglycine, 0.9184; Aminoadipic acid, GABA, 0.9479; Aminoadipic acid, Homoarginine, 0.9219; Aminoadipic acid, Kynurenine, 0.9271; Aminoadipic acid, N6-Acetyl-L-Lys, 0.9306; Aminoadipic acid, Putrescine, 0.9288; Aminoadipic acid, Serotonin, 0.9253; Aminoadipic acid, Spermidine, 0.9340; Aminoadipic acid, Spermine, 0.9201; Aminoadipic acid, ADMA, 0.9288; Aminoadipic acid, Homocitrulline, 0.9201; Aminoadipic acid, 3-Me-His, 0.9219; Aminoadipic acid, Hydroxyproline, 0.9306; Aminoadipic acid, Phosphoetanolamine, 0.9201; Aminoadipic acid, SDMA, 1.0000; Aminoadipic acid, N-Me-bABA, 0.9792; Aminoadipic acid, AC (13:1), 0.9531; Aminoadipic acid, EPA, 0.9427; bABA, bAiBA, 1.0000; bABA, Cadaverine, 1.0000; bABA, Ethylglycine, 0.9948; bABA, GABA, 0.9948; bABA, Homoarginine, 0.9965; bABA, Hypotaurine, 1.0000; bABA, Kynurenine, 0.9965; bABA, N6-Acetyl-L-Lys, 1.0000; bABA, Putrescine, 0.9965; bABA, Serotonin, 0.9948; bABA, Spermidine, 0.9948; bABA, Spermine, 0.9931; bABA, ADMA, 0.9948; bABA, Homocitrulline, 0.9965; bABA, 3-Me-His, 0.9983; bABA, Hydroxyproline, 1.0000; bABA, Phosphoetanolamine, 0.9983; bABA, SDMA, 1.0000; bABA, N-Me-bABA, 0.9965; bABA, AC (13:1), 0.9965; bABA, EPA, 0.9948; bAiBA, Cadaverine, 0.9618; bAiBA, GABA, 0.9375; bAiBA, Homoarginine, 0.9149; bAiBA, Kynurenine, 0.9219; bAiBA, N6-Acetyl-L-Lys, 0.9323; bAiBA, Putrescine, 0.9288; bAiBA, Serotonin, 0.9288; bAiBA, Spermidine, 0.9271; bAiBA, ADMA, 0.9219; bAiBA, Homocitrulline, 0.9201; bAiBA, 3-Me-His, 0.9288; bAiBA, Hydroxyproline, 0.9271; bAiBA, SDMA, 1.0000; bAiBA, N-Me-bABA, 0.9809; bAiBA, AC (13:1), 0.9566; bAiBA, EPA, 0.9427; Cadaverine, Ethylglycine, 0.9583; Cadaverine, GABA, 0.9653; Cadaverine, Homoarginine, 0.9566; Cadaverine, Hypotaurine, 0.9601; Cadaverine, Kynurenine, 0.9653; Cadaverine, N6-Acetyl-L-Lys, 0.9583; Cadaverine, Putrescine, 0.9792; Cadaverine, Serotonin, 0.9618; Cadaverine, Spermidine, 0.9601; Cadaverine, Spermine, 0.9566; Cadaverine, ADMA, 0.9549; Cadaverine, Homocitrulline, 0.9583; Cadaverine, 3-Me-His, 0.9601; Cadaverine, Hydroxyproline, 0.9601; Cadaverine, Phospho etanolamine, 0.9566; Cadaverine, SDMA, 1.0000; Cadaverine, N-Me-bABA, 0.9913; Cadaverine, AC (13:1), 0.9618; Cadaverine, EPA, 0.9618; Ethylglycine, GABA, 0.9392; Ethyl glycine, Homoarginine, 0.9167; Ethylglycine, Kynurenine, 0.9219; Ethylglycine, N6-Acetyl-L-Lys, 0.9288; Ethylglycine, Putrescine, 0.9271; Ethylglycine, Serotonin, 0.9201; Ethylglycine, Spermidine, 0.9288; Ethylglycine, Spermine, 0.9149; Ethylglycine, ADMA, 0.9253; Ethylglycine, Homocitrulline, 0.9167; Ethylglycine, 3-Me-His, 0.9184; Ethylglycine, Hydroxyproline, 0.9219; Ethylglycine, SDMA, 1.0000; Ethylglycine, N-Me-bABA, 0.9809; Ethylglycine, AC (13: 1), 0.9514; Ethylglycine, EPA, 0.9410; GABA, Homoarginine, 0.9444; GABA, Hypotaurine, 0.9410; GABA, Kynurenine, 0.9462; GABA, N6-Acetyl-L-Lys, 0.9358; GABA, Putrescine, 0.9462; GABA, Serotonin, 0.9392; GABA, Spermidine, 0.9462; GABA, Spermine, 0.9358; GABA, ADMA, 0.9444; GABA, Homocitrulline, 0.9323; GABA, 3-Me-His, 0.9444; GABA, Hydroxyproline, 0.9479; GABA, Phosphoetanolamine, 0.9410; GABA, SDMA, 1.0000; GABA, N-Me-bABA, 0.9826; GABA, AC (13:1), 0.9653; GABA, EPA, 0.9427; Homoarginine, Hypotaurine, 0.9149; Homoarginine, Kynurenine, 0.9271; Homoarginine, N6-Acetyl-L-Lys, 0.9306; Homoarginine, Putrescine, 0.9271; Homoarginine, Serotonin, 0.9184; Homoarginine, Spermidine, 0.9288; Homoarginine, Spermine, 0.9184; Homoarginine, ADMA, 0.9236; Homoarginine, Homocitrulline, 0.9184; Homoarginine, 3-Me-His, 0.9219; Homoarginine, Hydroxyproline, 0.9219; Homoarginine, Phosphoetanolamine, 0.9167; Homoarginine, SDMA, 1.0000; Homoarginine, N-Me-bABA, 0.9809; Homoarginine, AC (13:1), 0.9566; Homoarginine, EPA, 0.9392; Hypotaurine, Kynurenine, 0.9184; Hypotaurine, N6-Acetyl-L-Lys, 0.9340; Hypotaurine, Putrescine, 0.9306; Hypotaurine, Serotonin, 0.9184; Hypotaurine, Spermidine, 0.9323; Hypotaurine, ADMA, 0.9271; Hypotaurine, Homocitrulline, 0.9149; Hypotaurine, 3-Me-His, 0.9253; Hypotaurine, Hydroxyproline, 0.9219; Hypotaurine, SDMA, 1.0000; Hypotaurine, N-Me-bABA, 0.9826; Hypotaurine, AC (13:1), 0.9566; Hypotaurine, EPA, 0.9340; Kynurenine, N6-Acetyl-L-Lys, 0.9375; Kynurenine, PButrescine, 0.9392; Kynurenine, Serotonin, 0.9340; Kynurenine, Spermidine, 0.9375; Kynurenine, Spermine, 0.9236; Kynurenine, ADMA, 0.9410; Kynurenine, Homocitrulline, 0.9236; Kynurenine, 3-Me-His, 0.9271; Kynurenine, Hydroxyproline, 0.9323; Kynurenine, Phosphoetanolamine, 0.9253; Kynurenine, SDMA, 1.0000; Kynurenine, N-Me-bABA, 0.9861; Kynurenine, AC (13:1), 0.9566; Kynurenine, EPA, 0.9497; N6-Acetyl-L-Lys, Putrescine, 0.9375; N6-Acetyl-L-Lys, Serotonin, 0.9392; N6-Acetyl-L-Lys, Spermidine, 0.9340; N6-Acetyl-L-Lys, Spermine, 0.9306; N6-Acetyl-L-Lys, ADMA, 0.9392; N6-Acetyl-L-Lys, Homocitrulline, 0.9306; N6-Acetyl-L-Lys, 3-Me-His, 0.9375; N6-Acetyl-L-Lys, Hydroxyproline, 0.9392; N6-Acetyl-L-Lys, Phosphoetanolamine, 0.9253; N6-Acetyl-L-Lys, SDMA, 1.0000; N6-Acetyl-L-Lys, N-Me-bABA, 0.9826; N6-Acetyl-L-Lys, AC (13:1), 0.9601; N6-Acetyl-L-Lys, EPA, 0.9479; Putrescine, Serotonin, 0.9444; Putrescine, Spermidine, 0.9323; Putrescine, Spermine, 0.9271; Putrescine, ADMA, 0.9340; Putrescine, Homocitrulline, 0.9306; Putrescine, 3-Me-His, 0.9340; Putrescine, Hydroxyproline, 0.9323; Putrescine, Phosphoetanolamine, 0.9271; Putrescine, SDMA, 1.0000; Putrescine, N-Me-bABA, 0.9757; Putrescine, AC (13:1), 0.9549; Putrescine, EPA, 0.9340; Serotonin, Spermidine, 0.9444; Serotonin, Spermine, 0.9201; Serotonin, ADMA, 0.9340; Serotonin, Homocitrulline, 0.9236; Serotonin, 3-Me-His, 0.9288; Serotonin, Hydroxyproline, 0.9271; Serotonin, Phosphoetanolamine, 0.9149; Serotonin, SDMA, 1.0000; Serotonin, N-Me-bABA, 0.9774; Serotonin, AC (13:1), 0.9583; Serotonin, EPA, 0.9444; Spermidine, Spermine, 0.9375; Spermidine, ADMA, 0.9358; Spermidine, Homocitrulline, 0.9288; Spermidine, 3-Me-His, 0.9288; Spermidine, Hydroxyproline, 0.9288; Spermidine, Phosphoetanolamine, 0.9323; Spermidine, SDMA, 1.0000; Spermidine, N-Me-bABA, 0.9861; Spermidine, AC (13:1), 0.9566; Spermidine, EPA, 0.9462; Spermine, ADMA, 0.9236; Spermine, Homocitrulline, 0.9201; Spermine, 3-Me-His, 0.9236; Spermine, Hydroxyproline, 0.9253; Spermine, SDMA, 1.0000; Spermine, N-Me-bABA, 0.9809; Spermine, AC (13:1), 0.9531; Spermine, EPA, 0.9427; ADMA, Homocitrulline, 0.9340; ADMA, 3-Me-His, 0.9323; ADMA, Hydroxyproline, 0.9271; ADMA, Phosphoetanolamine, 0.9201; ADMA, SDMA, 1.0000; ADMA, N-Me-bABA, 0.9740; ADMA, AC (13:1), 0.9601; ADMA, EPA, 0.9410; Homocitrulline, 3-Me-His, 0.9219; Homocitrulline, Hydroxyproline, 0.9306; Homocitrulline, Phosphoetanolamine, 0.9271; Homocitrulline, SDMA, 1.0000; Homocitrulline, N-Me-bABA, 0.9844; Homocitrulline, AC (13:1), 0.9549; Homocitrulline, EPA, 0.9462; 3-Me-His, Hydroxyproline, 0.9340; 3-Me-His, Phosphoetanolamine, 0.9236; 3-Me-His, SDMA, 1.0000; 3-Me-His, N-Me-bABA, 0.9861; 3-Me-His, AC (13:1), 0.9583; 3-Me-His, EPA, 0.9531; Hydroxyproline, Phosphoetanolamine, 0.9201; Hydroxyproline, SDMA, 1.0000; Hydroxyproline, N-Me-bABA, 0.9861; Hydroxyproline, AC (13:1), 0.9549; Hydroxyproline, EPA, 0.9531; Phosphoetanolamine, SDMA, 1.0000; Phosphoetanolamine, N-Me-bABA, 0.9809; Phosphoetanolamine, AC (13:1), 0.9531; Phosphoetanolamine, EPA, 0.9392; SDMA, N-Me-bABA, 1.0000; SDMA, AC (13:1), 1.0000; SDMA, EPA, 1.0000; N-Me-bABA, AC (13:1), 0.9931; N-Me-bABA, EPA, 0.9809; AC (13:1), EPA, 0.9601

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An evaluating method comprising:
an evaluating step of evaluating a state of colorectal cancer for a subject to be evaluated using (i) a concentration value of at least one metabolite of bABA, N-Me-bABA, 1-Me-His, aABA, Aminoadipic acid, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, SDMA, Acylcarnitine (13:1), and EPA in blood of the subject or (ii) a value of a formula calculated using the concentration value of the metabolite and the formula including an explanatory variable to be substituted with the concentration value of the metabolite.

2. The evaluating method according to claim 1, wherein the evaluating step uses (i) the concentration value of the metabolite and a concentration value of at least one amino acid of Asn, His, Thr, Ala, Cit, Arg, Tyr, Val, Met, Lys, Trp, Gly, Pro, Orn, Ile, Leu, Phe, Ser, and Gln in blood of the subject or (ii) a value of the formula calculated using the concentration value of the metabolite, the concentration value of the amino acid, and the formula including an explanatory variable to be substituted with the concentration value of the amino acid.

3. The evaluating method according to claim 1 or 2, wherein the evaluating step is executed by a control unit that is included in an information processing apparatus.

4. An evaluating method comprising:
an evaluating step of evaluating a state of colorectal cancer for a subject to be evaluated, by calculating a value of a formula using (i) a concentration value of at least one metabolite of bABA, N-Me-bABA, 1-Me-His, aABA, Aminoadipic acid, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, SDMA, Acylcarnitine (13:1), and EPA in blood of the subject and (ii) the formula including an explanatory variable to be substituted with the concentration value of the metabolite.

5. An evaluating apparatus comprising a control unit, wherein the control unit includes an evaluating unit that evaluates a state of colorectal cancer for a subject to be evaluated using (i) a concentration value of at least one metabolite of bABA, N-Me-bABA, 1-Me-His, aABA, Aminoadipic acid, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, SDMA, Acylcarnitine (13:1), and EPA in blood of the subject or (ii) a value of a formula calculated using the concentration value of the metabolite and the formula including an explanatory variable to be substituted with the concentration value of the metabolite.

6. The evaluating apparatus according to claim 5, wherein the evaluating apparatus is connected communicatively via a network to a terminal apparatus that provides concentration data on the concentration value of the metabolite or the value of the formula,
the control unit further includes (i) a data-receiving unit that receives the concentration data of the subject or the value of the formula transmitted from the terminal apparatus and (ii) a result-sending unit that transmits an evaluation result obtained by the evaluating unit to the terminal apparatus,
the evaluating unit uses the concentration value of the metabolite included in the concentration data or the value of the formula received by the data-receiving unit.

7. An evaluating program product having a non-transitory tangible computer readable medium including programmed instructions for causing an information processing apparatus including a control unit to execute an evaluating method, wherein the evaluating method comprises an evaluating step of evaluating a state of colorectal cancer for a subject to be evaluated using (i) a concentration value of at least one metabolite of bABA, N-Me-bABA, 1-Me-His, aABA, Aminoadipic acid, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, SDMA, Acylcarnitine (13:1), and EPA in blood of the subject or (ii) a value of a formula calculated using the concentration value of the metabolite and the formula including an explanatory variable to be substituted with the concentration value of the metabolite.

8. An evaluating system comprising (I) an evaluating apparatus including a control unit and (II) a terminal apparatus including a control unit to provide (i) concentration data on a concentration value of at least one metabolite of bABA, N-Me-bABA, 1-Me-His, aABA, Aminoadipic acid, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, SDMA, Acylcarnitine (13:1), and EPA in blood of a subject to be evaluated or (ii) a value of a formula calculated using the concentration value of the metabolite and the formula including an explanatory variable to be substituted with the concentration value of the metabolite, wherein the evaluating apparatus and the terminal apparatus are connected to each other communicatively via a network,
wherein the control unit of the terminal apparatus includes:
a data-sending unit that transmits the concentration data of the subject or the value of the formula to the evaluating apparatus; and
a result-receiving unit that receives an evaluation result on a state of colorectal cancer for the subject transmitted from the evaluating apparatus, and
the control unit of the evaluating apparatus includes:
a data-receiving unit that receives the concentration data of the subject or the value of the formula transmitted from the terminal apparatus;
an evaluating unit that evaluates the state of colorectal cancer for the subject using the concentration value of the metabolite included in the concentration data of the subject or the value of the formula received by the data-receiving unit; and
a result-sending unit that transmits the evaluation result obtained by the evaluating unit to the terminal apparatus.

9. A terminal apparatus comprising a control unit, wherein the control unit includes a result-obtaining unit that obtains an evaluation result on a state of colorectal cancer for a subject to be evaluated,
wherein the evaluation result is the result of evaluating the state of colorectal cancer for the subject using (i) concentration data on a concentration value of at least one metabolite of bABA, N-Me-bABA, 1-Me-His, aABA, Aminoadipic acid, bAiBA, Cadaverine, Ethylglycine, GABA, Homoarginine, Hypotaurine, Kinurenine, N6-Acetyl-L-Lys, Putrescine, Serotonin, Spermidine, Spermine, ADMA, Homocitrulline, 3-Me-His, Hydroxyproline, Phosphoethanolamine, SDMA, Acylcarnitine (13:1), and EPA in blood of a subject to be evaluated or (ii) a value of a formula calculated using the concentration value of the metabolite and the formula including an explanatory variable to be substituted with the concentration value of the metabolite.
